(19) United States
(12) Patent Application Publication
(10) Pub. No.: US 2015/0011423 A1
(43) Pub. Date: Jan. 8, 2015

(54) MEANS AND METHODS FOR ASSESSING KIDNEY TOXICITY

(71) Applicants: Hennicke Kamp, Bischheim (DE); Tilmann B. Walk, Kleinmachnow (DE); Bennard van Ravenzwaay, Altrip (DE); Werner Mellert, Hassloch (DE); Eric Fabian, Speyer (DE); Volker Strauss, Bad Dürkheim (DE); Jan C. Wiemer, Berlin (DE); Ralf Looser, Berlin (DE); Michael Manfred Herold, Berlin (DE); Alexandre Prokoudine, Berlin (DE)

(72) Inventors: Hennicke Kamp, Bischheim (DE); Tilmann B. Walk, Kleinmachnow (DE); Bennard van Ravenzwaay, Altrip (DE); Werner Mellert, Hassloch (DE); Eric Fabian, Speyer (DE); Volker Strauss, Bad Dürkheim (DE); Jan C. Wiemer, Berlin (DE); Ralf Looser, Berlin (DE); Michael Manfred Herold, Berlin (DE); Alexandre Prokoudine, Berlin (DE)

(73) Assignee: BASF SE, Ludwigshafen (DE)

(21) Appl. No.: 14/344,277

(22) PCT Filed: Sep. 14, 2012
(86) PCT No.: PCT/IB2012/054790

§ 371 Date: Mar. 11, 2014

Related U.S. Application Data
(60) Provisional application No. 61/534,402, filed on Sep. 14, 2011.

(30) Foreign Application Priority Data
Sep. 14, 2011 (EP) 11181219.4
Oct. 28, 2011 (EP) 11187016.8

(51) Int. Cl. G01N 33/50; G01N 33/62; G01N 33/68; G01N 33/92; G01N 33/70

(52) U.S. Cl. CPC G01N 33/5014; G01N 33/92; G01N 33/70; G01N 33/6893; G01N 33/62; G01N 2800/347; G01N 2500/04
USPC 506/12; 436/173; 436/71; 436/111; 436/90; 436/108; 506/9; 435/7.92; 436/501; 506/18; 422/82.01; 506/39

(57) ABSTRACT

The present invention pertains to the field of diagnostics for kidney toxicity and toxicological assessments for risk stratification of chemical compounds. Specifically, it relates to a method for diagnosing kidney toxicity. It also relates to a method for determining whether a compound is capable of inducing such kidney toxicity in a subject and to a method of identifying a drug for treating kidney toxicity. Furthermore, the present invention relates to a device and a kit for diagnosing kidney toxicity.

The present invention pertains to the field of diagnostics for kidney toxicity and toxicological assessments for risk stratification of chemical compounds. Specifically, it relates to a method for diagnosing kidney toxicity. It also relates to a method for determining whether a compound is capable of inducing such kidney toxicity in a subject and to a method of identifying a drug for treating kidney toxicity. Furthermore, the present invention relates to a device and a kit for diagnosing kidney toxicity.

The kidneys are paired organs with several functions and have three major anatomical areas: the cortex, the medulla, and the papilla. The renal cortex is the outermost region of the kidney and contains glomeruli, proximal and distal tubules, and peritubular capillaries. Cortical blood flow is high, the cortex receiving approximately 90% of the renal blood flow. Since blood-borne toxicants will be delivered preferentially to the cortex, they are more likely to affect cortical functions rather than those of medulla or papilla. The renal medulla is the middle portion and contains primarily loops of Henle, vasa recta, and collecting ducts. Although the medulla receives only about 6% of the renal blood flow, it may be exposed to high concentrations of toxicants within tubular structures. The papilla is the smallest anatomical portion of the kidney and receives only about 1% of the renal blood flow. Nevertheless, because the tubular fluid is maximally concentrated and luminal fluid is maximally reduced, the concentrations of potential toxicants in the papilla my be extremely high, leading to cellular injury in the papillary tubular and/or interstitial cells. The nephron is the functional unit of the kidney. The primary function of the renal system is the elimination of waste products, derived either from endogenous metabolism or from the metabolism of xenobiotics. The kidney also plays an important role in regulation of body homeostasis, regulating extracellular fluid volume, and electrolyte balance. Other functions of the kidney include the synthesis of hormones that affect metabolism. Renin, a hormone involved in the formation of angiotensin and aldosterone, is formed in the kidney as are several prostaglandins.

Several factors are involved in the sensitivity of the kidney to a number of toxicants, although the high renal blood flow and the increased concentration of excretory products following reabsorption of water from the tubular fluid are clearly of major importance. The kidneys comprise less than 1% of the body mass but they receive around 25% of the cardiac output. Thus significant amounts of exogenous chemicals and/or their metabolites are delivered to the kidney. A second important factor affecting the kidneys sensitivity to chemicals is its ability to concentrate the tubular fluid and, as a consequence, to concentrate any chemicals it contains. The transport characteristics of the renal tubules also contribute to the delivery of potentially toxic concentrations of chemicals to the cells. If a chemical is actively secreted from the blood into the tubular fluid, it will accumulate initially within the cells of the proximal tubule or, if it is reabsorbed from the tubular fluid, it will pass into the cells in relatively high concentration. The biotransformation of chemicals to reactive, and thus potentially toxic, metabolites is a key feature of nephrotoxicity. Many of the same activation reactions found in the liver are also found in the kidney and many toxicants can be activated in either organ, including acetaminophen, bromobenzene, chloroform, and carbon tetrachloride, thus having potential for either hepatotoxicity or nephrotoxicity. Some regions of the kidney have considerable levels of xenobiotic metabolizing enzymes, particularly cytochrome P450 in the pars recta of the proximal tubule, a region particularly susceptible to chemical damage. Since reactive metabolites are generally unstable, and therefore more or less transient, they are likely to interact with cellular macromolecular components close to the site of generation. Thus, although the activity of activation enzymes such as cytochrome P450 is lower in the kidney that in the liver, they are of greater importance in nephrotoxicity than those of the liver due to their proximity to site of action. As with toxicity in other organs the ultimate expression of a toxic end point is the result of a balance between the generation of reactive metabolites and their detoxication. Other examples of nephrotoxicants include heavy metals. Certain antibiotics, most notably the aminoglycosides, are known to be nephrotoxic.

The kidney represents an organ frequently affected by xenobiotics due to its unique functional and structural organization and its role in the control of body homeostasis and the elimination of xenobiotics. Kidney toxicity, also called nephrotoxicity, refers to a chemically-induced or -driven kidney damage. Due to the diversity of possible actions of nephrotoxins, the assessment of kidney toxicity is a rather complex process. The current methods usually comprise clinical investigations (e.g. ultrasonography), pathological and histopathological investigations as well as a biochemical analysis. However, such parameters are rather complex regulated and changes may sometimes occur even at rather progressed stages. Major drawbacks of the histopathological assessments are that they are invasive, and even when combined with the clinical pathology measurements that they are less reliable because they are in part based an the individual interpretations of toxicologist carrying out the investigations. Moreover, the aforementioned diseases and disorders arising as a consequence of nephrotoxin-induced kidney toxicity can be hardly distinguished from other causes of the diseases or disorders by the current clinical measures (see, e.g., Cohen A H (2006) Renal anatomy and basic concepts and methods in renal pathology, 3-17, in.; Fogo A B, Cohen A H, Jennette J C, Bruijn J A, Colvin R B (eds) Fundamentals of renal pathology, Springer, New York, N.Y., USA; Greaves P (1998) The urinary system, 89-125, in: Target organ pathology, a basic text, Turton J and Hooson J (eds) Taylor & Francis, London, United Kingdom, 1998; Hodgson E, Levi P E (2004) Chapter 15 Nephrotoxicity, 273-278, in: A textbook of modern toxicology, 3rd editions (Hodgson ed.), Wiley-VCH Verlag GmbH, Weinheim Germany; Lemley K V, Kriz W (1991) Kidney Internat. 39: 370-381; Molema G, Meijer DKF (2001, eds) Drug Targeting Organ-Specific Strategies, Chapter 5, 121-156, Wiley-VCH Verlag GmbH, Weinheim Germany; Verlander J (1998), Toxiocl. Pathol. 26: 1-17).

The importance of kidney toxicity may become apparent if one considers that kidney toxicity by now is one of the most common reasons for a drug to be withdrawn from the market. Moreover, chemical compounds which are used in any kind of industry in the European Community, e.g., will now need to comply with REACH (Registration, Evaluation and Authorisation of Chemicals). It will be understood that the potential of a chemical compound to induce kidney toxicity will be deemed as a high risk for the compound and, consequently, the compound will be available only for limited applications and when obeying high security standards.

Sensitive and specific methods for assessing the toxicological properties of a chemical compound and, in particular, kidney toxicity, in an efficient and reliable manner are not yet available but would, nevertheless, be highly appreciated.

Thus, the technical problem underlying the present invention could be seen as the provision of means and methods for complying with the aforementioned needs. The technical problem is solved by the embodiments characterized in the claims and described herein below.

Accordingly, the present invention relates to a method for diagnosing kidney toxicity comprising:
(a) determining the amount of at least one biomarker selected from any one of Tables 1a, 1b, 1c, 1d, 2a, 2b, 2c, 2d, 3a, 3b, 3c, 3d, 4a, 4b, 4c, 4d, 5a, 5b, 6a, 6b, 7a, 7b, 8a, 8b, 11a or 11b in a test sample of a subject suspected to suffer from kidney toxicity, and
   (b) comparing the amounts determined in step (a) to a reference, whereby kidney toxicity is to be diagnosed.

In a preferred embodiment of the aforementioned method said subject has been brought into contact with a compound suspected to be capable of inducing kidney toxicity.

The present invention also relates to a method of determining whether a compound is capable of inducing kidney toxicity in a subject comprising:
(a) determining in a sample of a subject which has been brought into contact with a compound suspected to be capable of inducing kidney toxicity the amount of at least one biomarker selected from any one of Tables 1a, 1b, 1c, 1d, 2a, 2b, 2c, 2d, 3a, 3b, 3c, 3d, 4a, 4b, 4c, 4d, 5a, 5b, 6a, 6b, 7a, 7b, 8a, 8b, 11a or 11b; and
(b) comparing the amounts determined in step (a) to a reference, whereby the capability of the compound to induce kidney toxicity is determined.

In a preferred embodiment of the aforementioned method said compound is at least one compound selected from the group consisting of: Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, and decalin.

In another preferred embodiment of the methods of the present invention said reference is derived from (i) a subject or group of subjects which suffers from kidney toxicity or (ii) a subject or group of subjects which has been brought into contact with at least one compound selected from the group consisting of: Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, and decalin. In a more preferred embodiment of said method essentially identical amounts for the biomarkers in the test sample and the reference are indicative for kidney toxicity.

In another preferred embodiment of the methods of the present invention said reference is derived from (i) a subject or group of subjects known to not suffer from kidney toxicity or (ii) a subject or group of subjects which has not been brought into contact with at least one compound selected from the group consisting of: Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, and decalin. In a more preferred embodiment of said methods amounts for the biomarkers which differ in the test sample in comparison to the reference are indicative for kidney toxicity.

In yet another embodiment of the methods of the present invention said reference is a calculated reference for the biomarkers for a population of subjects. In a more preferred embodiment of said methods amounts for the biomarkers which differ in the test sample in comparison to the reference are indicative for kidney toxicity.

The present invention also contemplates a method of identifying a substance for treating kidney toxicity comprising the steps of:
(a) determining in a sample of a subject suffering from kidney toxicity which has been brought into contact with a candidate substance suspected to be capable of treating kidney toxicity the amount of at least one biomarker selected from any one of Tables 1a, 1b, 1c, 1d, 2a, 2b, 2c, 2d, 3a, 3b, 3c, 3d, 4a, 4b, 4c, 4d, 5a, 5b, 6a, 6b, 7a, 7b, 8a, 8b, 11a or 11b; and
(b) comparing the amounts determined in step (a) to a reference, whereby a substance capable of treating kidney toxicity is to be identified.

In a preferred embodiment of the aforementioned method said reference is derived from (i) a subject or group of subjects which suffers from kidney toxicity or (ii) a subject or group of subjects which has been brought into contact with at least one compound selected from the group consisting of: Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, and decalin. In a more preferred embodiment of said method amounts for the biomarkers which differ in the test sample and the reference are indicative for a substance capable of treating kidney toxicity.

In another preferred embodiment of the aforementioned method said reference is derived from (i) a subject or group of subjects known to not suffer from kidney toxicity or (ii) a subject or group of subjects which has not been brought into contact with at least one compound selected from the group consisting of: Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, and decalin. In a more preferred embodiment of the said methods essentially identical amounts for the biomarkers in the test sample and the reference are indicative for a substance capable of treating kidney toxicity.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating kidney toxicity.

The present invention also relates to the use of at least one biomarker selected from any one of Tables 1a, 1b, 1c, 1d, 2a, 2b, 2c, 2d, 3a, 3b, 3c, 3d, 4a, 4b, 4c, 4d, 5a, 5b, 6a, 6b, 7a, 7b, 8a, 8b, 11a or 11b or a detection agent for the said biomarker for diagnosing kidney toxicity in a sample of a subject.

Moreover, the present invention relates to a device for diagnosing kidney toxicity in a sample of a subject suspected to suffer therefrom comprising:
(a) an analyzing unit comprising a detection agent for at least one biomarker selected from any one of Tables 1a, 1b, 1c, 1d, 2a, 2b, 2c, 2d, 3a, 3b, 3c, 3d, 4a, 4b, 4c, 4d, 5a, 5b, 6a, 6b, 7a, 7b, 8a, 8b, 11a or 11b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby kidney toxicity is diagnosed.

In a preferred embodiment of the device of the invention said stored reference is a reference derived from a subject or a group of subjects known to suffer from kidney toxicity or a subject or group of subjects which has been brought into contact with at least one compound selected from the group consisting of Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, and decalin and said data processor executes instructions for comparing the amount of the at least one biomarker determined by the analyzing unit to the stored reference, wherein an essentially identical amount of the at least one biomarker in the test sample in comparison to the reference is indicative for the presence of kidney toxicity or wherein an amount of the at least one biomarker in the test sample which differs in comparison to the reference is indicative for the absence of kidney toxicity.

In another preferred embodiment of the device of the invention said stored reference is a reference derived from a subject or a group of subjects known to not suffer from kidney toxicity or a subject or group of subjects which has not been brought into contact with at least one compound selected from the group consisting of Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c Tricresyl phosphate, 1,1,2,2-Tetrachloroethane, 2,2,4-trimethylpentane, D-limonene, and decalin and said data processor executes instructions for comparing the amount of the at least one biomarker determined by the analyzing unit to the stored reference, wherein an amount of the at least one biomarker in the test sample which differs in comparison to the reference is indicative for the presence of kidney toxicity or wherein an essential identical amount of the at least one biomarker in the test sample in comparison to the reference is indicative for the absence of kidney toxicity.

Further, the present invention relates to a kit for diagnosing kidney toxicity comprising a detection agent for the at least one biomarker selected from any one of Tables 1a, 1b, 1c, 1d, 2a, 2b, 2c, 2d, 3a, 3b, 3c, 3d, 4a, 4b, 4c, 4d, 5a, 5b, 6a, 6b, 7a, 7b, 8a, 8b, 11a or 11b and standards for the at least one biomarker the concentration of which is derived from a subject or a group of subjects known to suffer from kidney toxicity or derived from a subject or a group of subjects known to not suffer from kidney toxicity.

In particular the present invention contemplates also the following specific methods, uses, devices and kits.

The following definitions and explanations apply mutatis mutandis to all the previous embodiments of the present invention as well as the embodiments described in the following.

The methods referred to in accordance with the present invention may essentially consist of the aforementioned steps or may include further steps. Further steps may relate to sample pre-treatment or evaluation of the diagnostic results obtained by the methods. Preferred further evaluation steps are described elsewhere herein. The methods may partially or entirely be assisted by automation. For example, steps pertaining to the determination of the amount of a biomarker can be automated by robotic and automated reader devices. Likewise, steps pertaining to a comparison of amounts can be automated by suitable data processing devices, such as a computer, comprising a program code which when being executed carries out the comparison automatically. A reference in such a case will be provided from a stored reference, e.g., from a database. It is to be understood that the method is, preferably, a method carried out ex vivo on a sample of a subject, i.e. not practised on the human or animal body.

The term “diagnosing” as used herein refers to assessing the probability according to which a subject is suffering from a condition, such as intoxication, disease or disorder referred to herein, or has a predisposition for such a condition. Diagnosis of a predisposition may sometimes be referred to as prognosis or prediction of the likelihood that a subject will develop the condition within a predefined time window in the future. As will be understood by those skilled in the art, such an assessment, although preferred to be, may usually not be correct for 100% of the subjects to be diagnosed. The term, however, requires that a statistically significant portion of subjects can be identified as suffering from the condition or having a predisposition for the condition. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, 0.05.

Diagnosing according to the present invention also includes monitoring, confirmation, and classification of a condition or its symptoms as well as a predisposition therefor. Monitoring refers to keeping track of an already diagnosed condition or predisposition. Monitoring encompasses, e.g., determining the progression of the condition or predisposition, determining the influence of a particular treatment on the progression of the condition or the influence of prophylactic measures such as a prophylactic treatment or diet on the development of the condition in a subject having a predisposition. Confirmation relates to the strengthening or substantiating a diagnosis of the condition or a predisposition for the condition already determined using other indicators or markers. Classification relates to (i) allocating the condition into different classes, e.g., corresponding to the strength of the symptoms accompanying the condition, or (ii) differentiating between different stages, disease or disorders accompanying the condition. A predisposition for the condition can be classified based on the degree of the risk, i.e. the probability according to which a subject will develop the condition later. Moreover, classification also, preferably, includes allocating a mode of action to a compound to be tested by the methods of the present invention. Specifically, the methods of the present invention allow for determination of a specific mode of action of a compound for which such mode of action is not yet known. This is, preferably, achieved by comparing the amount determined for the at least one biomarker or a biomarker profile representative for said compound to the amount of the biomarker or biomarker profile determined for a compound for which the mode of action is known as a reference. The classification of the mode of action allows an even more reliable assessment of toxicity of a compound because the molecular targets of the compound are identified.

The term “kidney toxicity” as used herein relates to any damage or impairment of the kidney which results in an impaired kidney function, in particular, impaired tubular or glomerular function. Preferably, affected by kidney toxicity are the excretion related functions of the kidney. Preferably, kidney toxicity as used herein is induced by or is the result of the administration of a chemical compound or drug, i.e. so-called toxin-induced kidney toxicity. More preferably, kidney toxicity is accompanied by an impaired renal tubular function. In particular, the proximal tubules are affected. Most preferably, the function of the P450 detoxification enzymes located in the pars recta of a proximal tubulus will become affected by kidney toxic compounds as referred to herein.

The symptoms and clinical signs of the aforementioned manifestations of kidney toxicity are well known to the person skilled in the art and are described in detail in standard books of toxicology, e.g., H. Marquardt, S. G. Schafer, R. O. McClellan, F. Welsch (eds.), “Toxicology”, Chapter 14: The Kidney, pp. 297-330 1999, Academic Press, London. Chapter 13: The Liver, 1999, Academic Press, London.

Preferred aspects of kidney toxicity involve impaired diuresis, glomerular-tubular defects, tubular defects, impaired excretion of weak acids, tubular necrosis, ACE inhibitor induced like defects such as renal impairment or renal failure, interstitial nephritis, alpha 2 u globulin-nephropathy, and/or direct tubular defects.

Biomarkers which are, preferably, to be determined for diagnosing the impaired diuresis as an aspect of kidney toxicity are those listed in Table 1a, 1b, 1c, and 1d.

Biomarkers which are, preferably, to be determined for diagnosing the glomerular-tubular defects an aspect of kidney toxicity are those listed in Table 2a, 2b, 2c, and 2d.

Biomarkers which are, preferably, to be determined for diagnosing the tubular defects as an aspect of kidney toxicity are those listed in Table 3a, 3b, 3c, and 3d.

Biomarkers which are, preferably, to be determined for diagnosing the impaired excretion of weak acids as an aspect of kidney toxicity are those listed in Table 4a, 4b, 4c, and 4d.

Biomarkers which are, preferably, to be determined for diagnosing the tubular necrosis as an aspect of kidney toxicity are those listed in Table 5a and 5b.

Biomarkers which are, preferably, to be determined for diagnosing the ACE inhibitor induced like defects as an aspect of kidney toxicity are those listed in Table 6a and 6b.

Biomarkers which are, preferably, to be determined for diagnosing the interstitial nephritis as an aspect of kidney toxicity are those listed in Table 7a and 7b.

Biomarkers which are, preferably, to be determined for diagnosing the direct tubular defects as an aspect of kidney toxicity are those listed in Table 8a and 8b.

Biomarkers which are, preferably, to be determined for diagnosing the alpha 2 u globulin-nephropathy as an aspect of kidney toxicity are those listed in Table 11a and 11b.

It was found in accordance with the present invention that a combination of more than one of the biomarkers listed in the Tables further strengthen the diagnosis since each of the biomarkers is an apparently statistically independent predictor for the diagnosis. Moreover, the specificity for kidney toxicity is also significantly increased since influences from other tissues on the marker abundance are counterbalanced. Thus, the term “at least one” as used herein, preferably, refers to a combination of at least 2, at least 3, at least 4, at least 5, at least 6, at least 7, at least 8, at least 9 or at least 10 of the biomarkers referred to in any one of the accompanying Tables. Preferably, all biomarkers recited in any one of the Tables are to be determined in combination in accordance with the methods of the present invention.

Thus, preferably, the at least one biomarker is at least one biomarker selected from the aforementioned group or the at least one biomarker is a combination of biomarkers consisting or comprising the aforementioned group of biomarkers. The aforementioned biomarkers and combinations of biomarkers have been identified as key biomarkers having a particular high diagnostic value as described in more detail in the accompanying Examples.

Furthermore, other biomarkers or clinical parameters including known metabolites, genetic mutations, transcript and/or protein amounts or enzyme activities may still be determined in addition. Such, additional clinical or biochemical parameters which may be determined in accordance with the method of the present invention are well known in the art.

The term “biomarker” as used herein refers to a chemical compound whose presence or concentration in a sample is indicative for the presence or absence or strength of a condition, preferably, kidney toxicity as referred to herein. The chemical compound is, preferably, a metabolite or an analyte derived therefrom. An analyte is a chemical compound which can be identical to the actual metabolite found in an organism. However, the term also includes derivatives of such metabolites which are either endogenously generated or which are generated during the isolation or sample pre-treatment or as a result of carrying out the methods of the invention, e.g., during the purification and/or determination steps. In specific cases the analyte is further characterized by chemical properties such as solubility. Due to the said properties, the analyte may occur in polar or lipid fractions obtained during the purification and/or determination process. Thus, chemical properties and, preferably, the solubility shall result in the occurrence of an analyte in either polar or lipid fractions obtained during the purification and/or determination process. Accordingly, the said chemical properties and, in particular the solubility taken into account as the occurrence of an analyte in either polar or lipid fractions obtained during the purification and/or determination process shall further characterize the analyte and assist in its identification. Details on how these chemical properties can be determined and taken into account are found in the accompanying Examples described below. Preferably, the analyte represents the metabolite in a qualitative and quantitative manner and, thus, allows inevitably concluding on the presence or absence or the amount of the metabolite in a subject or at least in the test sample of said subject. Biomarker, analyte and metabolite are referred to herein in the singular but also include the plurals of the terms, i.e. refer to a plurality of biomarker, analyte or metabolite molecules of the same molecular species. Moreover, a biomarker according to the present invention is not necessarily corresponding to one molecular species. Rather, the biomarker may comprise stereoisomers or enantiomers of a compound. Further, a biomarker can also represent the sum of isomers of a biological class of isomeric molecules. Said isomers shall exhibit identical analytical characteristics in some cases and are, therefore, not distinguishable by various analytical methods including those applied in the accompanying Examples described below. However, the isomers will share at least identical sum formula parameters and, thus, in the case of, e.g., lipids an identical chain length and identical numbers of double bonds in the fatty acid and/or sphingo base moieties

The term “test sample” as used herein refers to samples to be used for the diagnosis of kidney toxicity by the methods of the present invention. Preferably, said test sample is a biological sample. Samples from biological sources (i.e. biological samples) usually comprise a plurality of metabolites. Preferred biological samples to be used in the method of the present invention are samples from body fluids, preferably, blood, plasma, serum, saliva, bile, urine or cerebrospinal fluid, or samples derived, e.g. by biopsy, from cells, tissues or organs, preferably from the liver. More preferably, the sample is a blood, plasma or serum sample, most preferably, a plasma sample. Biological samples are derived from a subject as specified elsewhere herein. Techniques for obtaining the aforementioned different types of biological samples are well known in the art. For example, blood samples may be obtained by blood taking while tissue or organ samples are to be obtained, e.g. by biopsy.

The aforementioned samples are, preferably, pre-treated before they are used for the methods of the present invention. As described in more detail below, said pre-treatment may include treatments required to release or separate the compounds or to remove excessive material or waste. Suitable techniques comprise centrifugation, extraction, fractioning, ultra-filtration, protein precipitation followed by filtration and purification and/or enrichment of compounds. Moreover, other pre-treatments are carried out in order to provide the compounds in a form or concentration suitable for compound analysis. For example, if gas-chromatography coupled mass spectrometry is used in the method of the present invention, it will be required to derivatize the compounds prior to the said gas chromatography. Suitable and necessary pre-treatments depend on the means used for carrying out the method of the invention and are well known to the person skilled in the art. Pre-treated samples as described before are also comprised by the term “sample” as used in accordance with the present invention.

The term “subject” as used herein relates to animals, preferably to mammals such as mice, rats, guinea pigs, rabbits, hamsters, pigs, sheep, dogs, cats, horses, monkeys, or cows and, also preferably, to humans. More preferably, the subject is a rodent and, most preferably, a rat. Other animals which may be diagnosed applying the methods of the present invention are fishes, birds or reptiles. Preferably, said subject was in or has been brought into contact with a compound suspected to be capable of inducing kidney toxicity. A subject which has been brought into contact with a compound suspected to induce kidney toxicity may, e.g., be a laboratory animal such as a rat which is used in a screening assay for, e.g., toxicity of compounds. A subject suspected to have been in contact with a compound capable of inducing kidney toxicity may be also a subject to be diagnosed for selecting a suitable therapy. Preferably, a compound capable of inducing kidney toxicity as used herein is Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, or decalin.

Preferably, the at least one biomarker to be determined by the methods of the present invention is selected from any one of Tables 1a, 1b, 2a, 2b, 3a, 3b, 4a, 4b, 5a or 5b if the subject is a female.

Preferably, the at least one biomarker to be determined by the methods of the present invention is selected from any one of Tables 1c, 1d, 2c, 2d, 3c, 3d, 4c, 4d, 6a, 6b, 7a, 7b, 8a, 8b, 11a or 11b if the subject is a male.

A preferred group or combination of biomarkers which are, preferably, to be determined for diagnosing the impaired diuresis as an aspect of kidney toxicity encompasses or essentially consists of at least the following biomarkers 18-Hydroxy-11-deoxycorticosterone, Creatinine, Valine, trans-4-Hydroxyproline, and Proline for female subjects and Choline plasmalogen No 02, Sphingomyelin (d18:2,C16:0), Choline plasmalogen No 03, Threonine, and Ceramide (d18:1,C24:1) for male subjects. More preferably, the aforementioned biomarkers are changed with respect to a reference which has not been brought into contact with Caffeine, Furosemide, Lisinopril, Theobromine or Theophylline or which does not suffer from kidney toxicity as indicated in the accompanying Tables, below.

A preferred group or combination of biomarkers which are, preferably, to be determined for diagnosing the glomerular-tubular defects an aspect of kidney toxicity encompasses or essentially consists of at least the following biomarkers Creatine, Glucosamine, Mannosamine, Elaidic acid (C18:trans[9]1), and 3-Indoxylsulfate for female subjects and Threonic acid, Cysteine, Lysophosphatidylcholine (C18:2), Metanephrine, and Creatine for male subjects. More preferably, the aforementioned biomarkers are changed with respect to a reference which has not been brought into contact with Captopril, Cyclosporin A, Penicillamine, or Tricresyl phosphate or which does not suffer from kidney toxicity as indicated in the accompanying Tables, below.

A preferred group or combination of biomarkers which are, preferably, to be determined for diagnosing the tubular defects as an aspect of kidney toxicity encompasses or essentially consists of at least the following biomarkers Creatine, Lysophosphatidylcholine (C18:1), Indole-3-acetic acid, Histidine, and Glycerol, polar fraction for female subjects and Pseudouridine, Taurine, TAG (C18:1,C18:2), Allantoin, and Kynurenic acid for male subjects. More preferably, the aforementioned biomarkers are changed with respect to a reference which has not been brought into contact with Beta-ionone, Carboplatin, or Furosemide, Hexachlorobutadiene or which does not suffer from kidney toxicity as indicated in the accompanying Tables, below.

A preferred group or combination of biomarkers which are, preferably, to be determined for diagnosing the impaired excretion of weak acids as an aspect of kidney toxicity encompasses or essentially consists of at least the following biomarkers Proline, Lysophosphatidylcholine (C18:2), Indole-3-lactic acid, gamma-Linolenic acid (C18:cis[6,9,12]3), and trans-4-Hydroxyproline for female subjects and Lysophosphatidylcholine (C18:0), Phosphatidylcholine (C16:0,C20:5), Methionine, Indole-3-lactic acid, and Nervonic acid (C24:cis[15]1) for male subjects. More preferably, the aforementioned biomarkers are changed with respect to a reference which has not been brought into contact with Dichlorprop-p, MCPA, Mecoprop-p, Pentachlorophenol, or Probenecid, or which does not suffer from kidney toxicity as indicated in the accompanying Tables, below.

A preferred group or combination of biomarkers which are, preferably, to be determined for diagnosing the tubular necrosis as an aspect of kidney toxicity encompasses or essentially consists of at least the following biomarkers Hippuric acid, Docosahexaenoic acid (C22:cis[4,7,10,13,16,19]6), Leucine, Docosapentaenoic acid (C22:cis[7,10,13,16,19]5), and Lactate for female subjects. More preferably, the aforementioned biomarkers are changed with respect to a reference which has not been brought into contact with Amphotericin B, Hexachlorobutadiene, or Tobramycin s.c., or which does not suffer from kidney toxicity as indicated in the accompanying Tables, below.

A preferred group or combination of biomarkers which are, preferably, to be determined for diagnosing the ACE inhibitor induced like defects as an aspect of kidney toxicity encompasses or essentially consists of at least the following biomarkers Lysine, Glycine, Cytosine, 1,5-Anhydrosorbitol, and Glutamate for male subjects. More preferably, the aforementioned biomarkers are changed with respect to a reference which has not been brought into contact with Lisinopril or Ramipril, Theobromine, Theophylline, Tobramycin s.c., or Tricresyl phosphate or which does not suffer from kidney toxicity as indicated in the accompanying Tables, below.

A preferred group or combination of biomarkers which are, preferably, to be determined for diagnosing the interstitial nephritis as an aspect of kidney toxicity encompasses or essentially consists of at least the following biomarkers Lignoceric acid (C24:0), Creatine, Serine, Threonine, and Eicosanoic acid (C20:0) for male subjects. More preferably, the aforementioned biomarkers are changed with respect to a reference which has not been brought into contact with Dipyrone, Ethylbenzene, or Lithocholic acid or which does not suffer from kidney toxicity as indicated in the accompanying Tables, below.

A preferred group or combination of biomarkers which are, preferably, to be determined for diagnosing the direct tubular defects as an aspect of kidney toxicity encompasses or essentially consists of at least the following biomarkers Lysophosphatidylethanolamine (C22:0), Leucine, Oleic acid (C18:cis[9]1), TAG No 02, and Glycerol-3-phosphate, polar fraction for male subjects. More preferably, the aforementioned biomarkers are changed with respect to a reference which has not been brought into contact with Hexachlorobutadiene or Hydroquinone or which does not suffer from kidney toxicity as indicated in the accompanying Tables, below.

The term “determining the amount” as used herein refers to determining at least one characteristic feature of the biomarker, i.e. the metabolite or analyte. Characteristic features in accordance with the present invention are features which characterize the physical and/or chemical properties including biochemical properties of a biomarker. Such properties include, e.g., molecular weight, viscosity, density, electrical charge, spin, optical activity, colour, fluorescence, chemoluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a biomarker by standard operations, e.g., mathematical calculations such as multiplication, division or logarithmic calculus. Most preferably, the at least one characteristic feature allows the determination and/or chemical identification of the biomarker and its amount. Accordingly, the characteristic value, preferably, also comprises information relating to the abundance of the biomarker from which the characteristic value is derived. For example, a characteristic value of a biomarker may be a peak in a mass spectrum. Such a peak contains characteristic information of the biomarker, i.e. the m/z (mass to charge ratio) information, as well as an intensity value being related to the abundance of the said biomarker (i.e. its amount) in the sample.

As discussed before, the at least one biomarker to be determined in accordance with the methods of the present invention may be, preferably, determined quantitatively or semi-quantitatively. For quantitative determination, either the absolute or precise amount of the biomarker will be determined or the relative amount of the biomarker will be determined based on the value determined for the characteristic feature(s) referred to herein above. The relative amount may be determined in a case were the precise amount of a biomarker can or shall not be determined. In said case, it can be determined whether the amount in which the biomarker is present is enlarged or diminished with respect to a second sample comprising said biomarker in a second amount. Quantitatively analysing a biomarker, thus, also includes what is sometimes referred to as semi-quantitative analysis of a biomarker.

Moreover, determining as used in the methods of the present invention, preferably, includes using a compound separation step prior to the analysis step referred to before. Preferably, said compound separation step yields a time resolved separation of the at least one biomarker comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or GC are chromatographic techniques to be envisaged by the methods of the present invention. Suitable devices for such determination of biomarkers are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Most preferably, LC-MS and/or GC-MS are used as described in detail below. Said techniques are disclosed in, e.g., Nissen 1995, Journal of Chromatography A, 703: 37-57, U.S. Pat. No. 4,540,884 or U.S. Pat. No. 5,397,894, the disclosure content of which is hereby incorporated by reference. As an alternative or in addition to mass spectrometry techniques, the following techniques may be used for compound determination: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado. The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

Moreover, the biomarker can also be determined by a specific chemical or biological assay. Said assay shall comprise means which allow for specifically detecting the biomarker in the sample. Preferably, said means are capable of specifically recognizing the chemical structure of the biomarker or are capable of specifically identifying the biomarker based on its capability to react with other compounds or its capability to elicit a response in a biological read out system (e.g., induction of a reporter gene). Means which are capable of specifically recognizing the chemical structure of a biomarker are, preferably, detection agents which specifically bind to the biomarker, more preferably, antibodies or other proteins which specifically interact with chemical structures, such as receptors or enzymes, or aptameres. Specific antibodies, for instance, may be obtained using the biomarker as antigen by methods well known in the art. Antibodies as referred to herein include both polyclonal and monoclonal antibodies, as well as fragments thereof, such as Fv, Fab and F(ab)₂ fragments that are capable of binding the antigen or hapten. The present invention also includes humanized hybrid antibodies wherein amino acid sequences of a non-human donor antibody exhibiting a desired antigen-specificity are combined with sequences of a human acceptor antibody. Moreover, encompassed are single chain antibodies. The donor sequences will usually include at least the antigen-binding amino acid residues of the donor but may comprise other structurally and/or functionally relevant amino acid residues of the donor antibody as well. Such hybrids can be prepared by several methods well known in the art. Suitable proteins which are capable of specifically recognizing the metabolite are, preferably, enzymes which are involved in the metabolic conversion of the said biomarker. Said enzymes may either use the biomarker, e.g., a metabolite, as a substrate or may convert a substrate into the biomarker, e.g., metabolite. Moreover, said antibodies may be used as a basis to generate oligopeptides which specifically recognize the biomarker. These oligopeptides shall, for example, comprise the enzyme's binding domains or pockets for the said biomarker. Suitable antibody and/or enzyme based assays may be RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA) or solid phase immune tests. Aptameres which specifically bind to the biomarker can be generated by methods well known in the art (Ellington 1990, Nature 346:818-822; Vater 2003, Curr Opin Drug Discov Devel 6(2): 253-261). Moreover, the biomarker may also be identified based on its capability to react with other compounds, i.e. by a specific chemical reaction. Further, the biomarker may be determined in a sample due to its capability to elicit a response in a biological read out system. The biological response shall be detected as read out indicating the presence and/or the amount of the metabolite comprised by the sample. The biological response may be, e.g., the induction of gene expression or a phenotypic response of a cell or an organism.

The term “reference” refers to values of characteristic features of the at least one biomarker and, preferably, values indicative for an amount of the said biomarker which can be correlated to kidney toxicity.

Such references are, preferably, obtained from a sample derived from a subject or group of subjects which suffer from kidney toxicity or from a sample derived from a subject or group of subjects which have/has been brought into contact with Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, or decalin. A subject or group of subjects may be brought into contact with the said compounds by each topic or systemic administration mode as long as the compounds become bioavailable. Preferred administration modes are described in the accompanying Examples, below. Alternatively, but nevertheless also preferred, the reference may be obtained from sample derived from a subject or group of subjects which has not been brought into contact with Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, or decalin or a healthy subject or group of such subjects with respect to kidney toxicity and, more preferably, other diseases as well.

The reference may be determined as described hereinabove for the amounts of the biomarkers. In particular, a reference is, preferably, obtained from a sample of a group of subjects as referred to herein by determining the relative or absolute amounts of each of the at least one biomarker(s) in samples from each of the individuals of the group separately and subsequently determining a median or average value for said relative or absolute amounts or any parameter derived therefrom by using statistical techniques referred to elsewhere herein. Alternatively, the reference may be, preferably, obtained by determining the relative or absolute amount for each of the at least one biomarker in a sample from a mixture of samples of the group of subjects as referred to herein. Such a mixture, preferably, consists of portions of equal volume from samples obtained from each of the individuals of the said group.

Moreover, the reference, also preferably, could be a calculated reference, most preferably the average or median value, for the relative or absolute amount for each of the at least one biomarker derived from a population of individuals. Said population of individuals is the population from which the subject to be investigated by the method of the present invention originates. However, it is to be understood that the population of subjects to be investigated for determining a calculated reference, preferably, either consist of apparently healthy subjects (e.g. untreated) or comprise a number of apparently healthy subjects which is large enough to be statistically resistant against significant average or median changes due to the presence of the test subject(s) in the said population. The absolute or relative amounts of the at least one biomarker of said individuals of the population can be determined as specified elsewhere herein. How to calculate a suitable reference value, preferably, the average or median, is well known in the art. Other techniques for calculating a suitable reference include optimization using receiver operating characteristics (ROC) curve calculations which are also well known in the art and which can be performed for an assay system having a given specificity and sensitivity based on a given cohort of subjects without further ado. The population or group of subjects referred to before shall comprise a plurality of subjects, preferably, at least 5, 10, 50, 100, 1,000 or 10,000 subjects up to the entire population. More preferably, the group of subjects referred to in this context is a group of subjects having a size being statistically representative for a given population, i.e. a statistically representative sample. It is to be understood that the subject to be diagnosed by the methods of the present invention and the subjects of the said plurality of subjects are of the same species and, preferably, of the same gender.

More preferably, the reference will be stored in a suitable data storage medium such as a database and are, thus, also available for future diagnoses. This also allows efficiently diagnosing predisposition for kidney toxicity because suitable reference results can be identified in the database once it has been confirmed (in the future) that the subject from which the corresponding reference sample was obtained (indeed) developed kidney toxicity.

The term “comparing” refers to assessing whether the amount of the qualitative or quantitative determination of the at least one biomarker is identical to a reference or differs therefrom.

In case the reference results are obtained from a sample derived from a subject or group of subjects suffering from kidney toxicity or a subject or group of subjects which has been brought into contact with Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, or decalin, kidney toxicity can be diagnosed based on the degree of identity or similarity between the amounts obtained from the test sample and the aforementioned reference, i.e. based on an identical qualitative or quantitative composition with respect to the at least one biomarker. Identical amounts include those amounts which do not differ in a statistically significant manner and are, preferably, within at least the interval between 1^{st} and 99^{th} percentile, 5^{th} and 95^{th} percentile, 10^{th} and 90^{th} percentile, 20^{th} and 80^{th} percentile, 30^{th} and 70^{th} percentile, 40^{th} and 60^{th} percentile of the reference, more preferably, the 50^{th}, 60^{th}, 70^{th}, 80^{th}, 90^{th} or 95^{th} percentile of the reference. A reference obtained from a sample derived from a subject or group of subjects suffering from kidney toxicity or a subject or group of subjects which has been brought into contact with Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, or decalin, can be applied in the methods of the present invention in order to diagnose kidney toxicity or for determining whether a compound is capable of inducing kidney toxicity in a subject. In such a case, preferably, an amount of the at least one biomarker which is essentially identical to the reference will be indicative for the presence of kidney toxicity or a compound which is capable of inducing kidney toxicity, while an amount of the at least one biomarker which differs from the reference will be indicative for the absence of kidney toxicity or a compound which is not capable of inducing kidney toxicity.

Moreover, a reference obtained from a sample derived from a subject or group of subjects suffering from kidney toxicity or a subject or group of subjects which has been brought into contact with Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, or decalin, can be applied for identifying a substance for treating kidney toxicity. In such a case, preferably, an amount of the at least one biomarker which differs from the reference will be indicative for a substance suitable for treating kidney toxicity, while an amount of the at least one biomarker which is essentially identical to the reference will be indicative for a substance which is not capable of treating kidney toxicity.

In case the reference results are obtained from a sample of a subject or group of subjects which has not been brought into contact with Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, or decalin or which does not suffer from kidney toxicity, said kidney toxicity can be diagnosed based on the differences between the test amounts obtained from the test sample and the aforementioned reference, i.e. differences in the qualitative or quantitative composition with respect to the at least one biomarker.

The same applies if a calculated reference as specified above is used.

The difference may be an increase in the absolute or relative amount of the at least one biomarker (sometimes referred to as up-regulation of the biomarker; see also Examples) or a decrease in either of said amounts or the absence of a detectable amount of the biomarker (sometimes referred to as down-regulation of the biomarker; see also Examples). Preferably, the difference in the relative or absolute amount is significant, i.e. outside of the interval between 45^{th} and 55^{th} percentile, 40^{th} and 60^{th} percentile, 30^{th} and 70^{th} percentile, 20^{th} and 80^{th} percentile, 10^{th} and 90^{th} percentile, 5^{th} and 95^{th} percentile, 1^{st} and 99^{th} percentile of the reference.

A reference obtained from a sample derived from a subject or group of subjects which has not been brought into contact with Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, or decalin or which does not suffer from kidney toxicity can be applied in the methods of the present invention in order to diagnose the kidney toxicity or for determining whether a compound is capable of inducing kidney toxicity in a subject. In such a case, preferably, an amount of the at least one biomarker which differs from the reference will be indicative for the presence of kidney toxicity or a compound which is capable of inducing kidney toxicity, while an amount of the at least one biomarker which is essentially identical to the reference will be indicative for the absence of kidney toxicity or a compound which is not capable of inducing kidney toxicity. Moreover, a reference obtained from a sample derived from a subject or group of subjects which has not been brought into contact with Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, or decalin or which does not suffer from kidney toxicity can be applied for identifying a substance for treating kidney toxicity. In such a case, preferably, an amount of the at least one biomarker which is essentially identical to the reference will be indicative for a substance suitable for treating kidney toxicity, while an amount of the at least one biomarker which differs from the reference will be indicative for a substance which is not suitable for treating kidney toxicity.

Preferred references are those referred to in the accompanying Tables or those which can be generated following the accompanying Examples. Moreover, relative differences, i.e. increases or decreases in the amounts for individual biomarkers, are preferably, those recited in the Tables below. Moreover, preferably, the extent of an observed difference, i.e. an increase or decrease, is preferably, an increase or decrease according to the factor indicated in the Tables, below.

Preferably, the at least one biomarker when selected from Tables 1a, 1c, 2a, 2c, 3a, 3c, 4a, 4c, 5a, 6a, 7a, 8a, or 11a is increased with respect to a reference which has not been brought into contact with Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, or decalin or which does not suffer from kidney toxicity and, more preferably, a reference as used for the Tables, below.

Preferably, the at least one biomarker when selected from Tables 1b, 1d, 2b, 2d, 3b, 3d, 4b, 4d, 5b, 6b, 7b, 8b, or 11b is decreased with respect to a reference which has not been brought into contact with Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, or decalin or which does not suffer from kidney toxicity and, more preferably, a reference as used for the Tables, below.

The comparison is, preferably, assisted by automation. For example, a suitable computer program comprising algorithm for the comparison of two different data sets (e.g., data sets comprising the values of the characteristic feature(s)) may be used. Such computer programs and algorithm are well known in the art. Notwithstanding the above, a comparison can also be carried out manually.

The term “substance for treating kidney toxicity” refers to compounds which may directly interfere with the biological mechanisms inducing kidney toxicity referred to elsewhere in this specification Alternatively, but also preferred the compounds may interfere with the development or progression of symptoms associated with the kidney toxicity. Substances to be identified by the method of the present invention may be organic and inorganic chemicals, such as small molecules, polynucleotides, oligonucleotides including siRNA, ribozymes or micro RNA molecules, peptides, polypeptides including antibodies or other artificial or biological polymers, such as aptameres. Preferably, the substances are suitable as drugs, pro-drugs or lead substances for the development of drugs or pro-drugs.

It is to be understood that if the methods of the present invention are to be used for identifying drugs for the therapy of kidney toxicity or for toxicological assessments of compounds (I.e. determining whether a compound is capable of inducing kidney toxicity), test samples of a plurality of subjects may be investigated for statistical reasons. Preferably, the metabolome within such a cohort of test subjects shall be as similar as possible in order to avoid differences which are caused, e.g., by factors other than the compound to be investigated. Subjects to be used for the said methods are, preferably, laboratory animals such as rodents and more preferably rats. It is to be understood further that the said laboratory animals shall be, preferably, sacrificed after completion of the methods of the present invention. All subjects of a cohort test and reference animals shall be kept under identical conditions to avoid any differential environmental influences. Suitable conditions and methods of providing such animals are described in detail in WO2007/014825. Said conditions are hereby incorporated by reference.

The methods of the present invention can be, preferably, implemented by the device of the present invention. A device as used herein shall comprise at least the aforementioned units. The units of the device are operatively linked to each other. How to link the units in an operating manner will depend on the type of units included into the device. For example, where means for automatically qualitatively or quantitatively determining the at least one biomarker are applied in an analyzing unit, the data obtained by said automatically operating unit can be processed by the evaluation unit, e.g., by a computer program which runs on a computer being the data processor in order to facilitate the diagnosis. Preferably, the units are comprised by a single device in such a case. However, the analyzing unit and the evaluation unit may also be physically separate. In such a case operative linkage can be achieved via wire and wireless connections between the units which allow for data transfer. A wireless connection may use Wireless LAN (WLAN) or the internet. Wire connections may be achieved by optical and non-optical cable connections between the units. The cables used for wire connections are, preferably, suitable for high throughput data transport

A preferred analyzing unit for determining at least one biomarker comprises a detection agent, such as an antibody, protein or aptamere which specifically recognizes the at least one biomarker as specified elsewhere herein, and a zone for contacting said detection agent with the sample to be tested. The detection agent may be immobilized on the zone for contacting or may be applied to the said zone after the sample has been loaded. The analyzing unit shall be, preferably, adapted for qualitatively and/or quantitatively determine the amount of complexes of the detection agent and the at least one biomarker. It will be understood that upon binding of the detection agent to the at least one biomarker, at least one measurable physical or chemical property of either the at least one biomarker, the detection agent or both will be altered such that the said alteration can be measured by a detector, preferably, comprised in the analyzing unit. However, where analyzing units such as test stripes are used, the detector and the analyzing units may be separate components which are brought together only for the measurement. Based on the detected alteration in the at least one measurable physical or chemical property, the analyzing unit may calculate an intensity value for the at least one biomarker as specified elsewhere herein. Said intensity value can then be transferred for further processing and evaluation to the evaluation unit. Most preferably, the amount of the at least one biomarker can be determined by ELISA, EIA, or RIA based techniques using a detection agent as specified elsewhere herein. Alternatively, an analyzing unit as referred to herein, preferably, comprises means for separating biomarkers, such as chromatographic devices, and means for biomarker determination, such as spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS and/or GC-MS is used in the analyzing unit referred to in accordance with the present invention.

The evaluation unit of the device of the present invention, preferably, comprises a data processing device or computer which is adapted to execute rules for carrying out the comparison as specified elsewhere herein. Moreover, the evaluation unit, preferably, comprises a database with stored references. A database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for kidney toxicity (e.g. a query search). Thus, if an identical or similar data set can be identified in the data collection, the test data set will be associated with kidney toxicity. The evaluation unit may also preferably comprise or be operatively linked to a further database with recommendations for therapeutic or preventive interventions or life style adaptations based on the established diagnosis of kidney toxicity. Said further database can be, preferably, automatically searched with the diagnostic result obtained by the evaluation unit in order to identify suitable recommendations for the subject from which the test sample has been obtained in order to treat or prevent kidney toxicity.

In a preferred embodiment of the device of the present invention, said stored reference is a reference derived from a subject or a group of subjects known to suffer from kidney toxicity or a subject or group of subjects which has been brought into contact with at least one compound selected from the group consisting of Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, and decalin and said data processor executes instructions for comparing the amount of the at least one biomarker determined by the analyzing unit to the stored reference, wherein an essentially identical amount of the at least one biomarker in the test sample in comparison to the reference is indicative for the presence of kidney toxicity or wherein an amount of the at least one biomarker in the test sample which differs in comparison to the reference is indicative for the absence of kidney toxicity.

In another preferred embodiment of the device of the present invention, said stored reference is a reference derived from a subject or a group of subjects known not to suffer from kidney toxicity or a subject or group of subjects which has not been brought into contact with at least one compound selected from the group consisting of Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, and decalin and said data processor executes instructions for comparing the amount of the at least one biomarker determined by the analyzing unit to the stored reference, wherein an amount of the at least one biomarker in the test sample which differs in comparison to the reference is indicative for the presence of kidney toxicity or wherein an essentially identical amount of the at least one biomarker in the test sample in comparison to the reference is indicative for the absence of kidney toxicity.

The device, thus, can also be used without special medical knowledge by medicinal or laboratory staff or patients, in particular when an expert system making recommendations is included. The device is also suitable for near-patient applications since the device can be adapted to a portable format.

The term “kit” refers to a collection of the aforementioned components, preferably, provided separately or within a single container. The container also comprises instructions for carrying out the method of the present invention. These instructions may be in the form of a manual or may be provided by a computer program code which is capable of carrying out the comparisons referred to in the methods of the present invention and to establish a diagnosis accordingly when implemented on a computer or a data processing device. The computer program code may be provided on a data storage medium or device such as an optical or magnetic storage medium (e.g., a Compact Disc (CD), CD-ROM, a hard disk, optical storage media, or a diskette) or directly on a computer or data processing device. A “standard” as referred to in connection with the kit of the invention is an amount of the at least one biomarker when present in solution or dissolved in a predefined volume of a solution resembles the amount of the at least one biomarker which is present (i) in a subject or a group of subjects known to suffer from kidney toxicity or a subject or group of subjects which has been brought into contact with at least one compound selected from the group consisting of Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, and decalin or (ii) derived from a subject or a group of subjects known to not suffer from therefrom or a subject or group of subjects which has not been brought into contact with at least one compound selected from the group consisting of Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, and decalin.

Advantageously, it has been found in the study underlying the present invention that the amount of at least one biomarker as specified herein allows for diagnosing kidney toxicity, specifically kidney toxicity induced by Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-Tetrachloroethane, 2,2,4-trimethylpentane, D-limonene, and decalin. The specificity and accuracy of the method will be even more improved by determining an increasing number or even all of the aforementioned biomarkers. A change in the quantitative and/or qualitative composition of the metabolome with respect to these specific biomarkers is indicative for kidney toxicity even before other signs of the said toxicity are clinically apparent. The morphological, physiological as well as biochemical parameters which are currently used for diagnosing kidney toxicity are less specific and less sensitive in comparison to the biomarker determination provided by the present invention. Thanks to the present invention, kidney toxicity of a compound can be more efficiently and reliably assessed. Moreover, based on the aforementioned findings, screening assays for drugs which are useful for the therapy of kidney toxicity are feasible. In general, the present invention contemplates the use of at least one biomarker in a sample of a subject selected from any one of the Tables 1a, 1b, 1c, 1d, 2a, 2b, 2c, 2d, 3a, 3b, 3c, 3d, 4a, 4b, 4c, 4d, 5a, 5b, 6a, 6b, 7a, 7b, 8a, 8b, 11a or 11b or a detection agent for said biomarker for diagnosing kidney toxicity, for determining whether a compound is capable of inducing kidney toxicity or for identifying a substance capable of treating kidney toxicity. Further, the present invention, in general, contemplates the use of the at least one biomarker in a sample of a subject or a detection agent therefor for identifying a subject being susceptible for a treatment of kidney toxicity. Preferred detection agents to be used in this context of the invention are those referred to elsewhere herein. Moreover, the methods of the present invention can be, advantageously, implemented into a device. Furthermore, a kit can be provided which allows for carrying out the methods.

The present invention also relates to a data collection comprising characteristic values for the biomarkers recited in any one of Tables 1a, 1b, 1c, 1d, 2a, 2b, 2c, 2d, 3a, 3b, 3c, 3d, 4a, 4b, 4c, 4d, 5a, 5b, 6a, 6b, 7a, 7b, 8a, 8b, 11a or 11b. The term “data collection” refers to a collection of data which may be physically and/or logically grouped together. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for kidney toxicity (e.g. a query search). Thus, if an identical or similar data set can be identified in the data collection, the test data set will be associated with kidney toxicity. Consequently, the information obtained from the data collection can be used to diagnose kidney toxicity based on a test data set obtained from a subject.

Moreover, the present invention pertains to a data storage medium comprising the said data collection. The term “data storage medium” as used herein encompasses data storage media which are based on single physical entities such as a CD, a CD-ROM, a hard disk, optical storage media, or a diskette. Moreover, the term further includes data storage media consisting of physically separated entities which are operatively linked to each other in a manner as to provide the aforementioned data collection, preferably, in a suitable way for a query search.

The present invention also relates to a system comprising
(a) means for comparing characteristic values of at least one biomarker of a sample operatively linked to
(b) the data storage medium of the present invention.

The term “system” as used herein relates to different means which are operatively linked to each other. Said means may be implemented in a single device or may be implemented in physically separated devices which are operatively linked to each other. The means for comparing characteristic values of the biomarker operate, preferably, based on an algorithm for comparison as mentioned before. The data storage medium, preferably, comprises the aforementioned data collection or database, wherein each of the stored data sets being indicative for kidney toxicity. Thus, the system of the present invention allows identifying whether a test data set is comprised by the data collection stored in the data storage medium. Consequently, the system of the present invention may be applied as a diagnostic means in diagnosing kidney toxicity. In a preferred embodiment of the system, means for determining characteristic values of biomakers of a sample are comprised. The term “means for determining characteristic values of biomarkers” preferably relates to the aforementioned devices for the determination of biomarkers such as mass spectrometry devices, ELISA devices, NMR devices or devices for carrying out chemical or biological assays for the analytes.

All references referred to above are herewith incorporated by reference with respect to their entire disclosure content as well as their specific disclosure content explicitly referred to in the above description.

The following Examples are merely for the purposes of illustrating the present invention. They shall not be construed, whatsoever, to limit the scope of the invention in any respect.

### EXAMPLES

### Example

### Biomarkers Associated with Kidney Toxicity

A group of each 5 male and female rats was dosed once daily with the indicated compounds (see Table 9, below for compounds, applied doses and administration details) over 28 days.

Each dose group in the studies consisted of five rats per sex. Additional groups of each 5 male and female animals served as controls. Before starting the treatment period, animals, which were 62-64 days old when supplied, were acclimatized to the housing and environmental conditions for 7 days. All animals of the animal population were kept under the same constant temperature (20-24±3° C.) and the same constant humidity (30-70%). The animals of the animal population were fed ad libitum. The food to be used was essentially free of chemical or microbial contaminants. Drinking water was also offered ad libitum. Accordingly, the water was free of chemical and microbial contaminants as laid down in the European Drinking Water Directive 98/83/EG. The illumination period was 12 hours light followed by 12 hours darkness (12 hours light, from 6:00 to 18:00, and 12 hours darkness, from 18:00 to 6:00). The studies were performed in an AAALAC-approved laboratory in accordance with the German Animal Welfare Act and the European Council Directive 86/609/EE. The test system was arranged according to the OECD 407 guideline for the testing of chemicals for repeated dose 28-day oral toxicity study in rodents. The test substances (compounds in Table 9 were dosed and administered as described in the table above.

In the morning of day 7, 14, and 28, blood was taken from the retroorbital venous plexus from fasted anaesthetized animals. From each animal, 1 ml of blood was collected with EDTA as anticoagulant. The samples were centrifuged for generation of plasma. All plasma samples were covered with a N2 atmosphere and then stored at −80° C. until analysis.

For mass spectrometry-based metabolite profiling analyses plasma samples were extracted and a polar and a non-polar (lipid) fraction was obtained. For GC-MS analysis, the non-polar fraction was treated with methanol under acidic conditions to yield the fatty acid methyl esters. Both fractions were further derivatised with O-methyl-hydroxyamine hydrochloride and pyridine to convert Oxo-groups to O-methyloximes and subsequently with a silylating agent before analysis. In LC-MS analysis, both fractions were reconstituted in appropriate solvent mixtures. HPLC was performed by gradient elution on reversed phase separation columns. Mass spectrometric detection which allows target and high sensitivity MRM (Multiple Reaction Monitoring) profiling in parallel to a full screen analysis was applied as described in WO2003073464.

Steroids and their metabolites were measured by online SPE-LC-MS (Solid phase extraction-LC-MS). Catecholamines and their metabolites were measured by online SPE-LC-MS as described by Yamada et al. (Yamada 2002, Journal of Analytical Toxicology, 26(1): 17-22))

Following comprehensive analytical validation steps, the data for each analyte were normalized against data from pool samples. These samples were run in parallel through the whole process to account for process variability. The significance of treatment group values specific for sex, treatment duration and metabolite was determined by comparing means of the treated groups to the means of the respective untreated control groups using WELCH-test and quantified with treatment ratios versus control and p-values.

The identification of the most important biomarkers per toxicity pattern was done by a ranking of the analytes in the tables below. Therefore the metabolic changes in reference treatments of a given pattern (shown in the Tables) were compared with changes of the same metabolite in other unrelated treatments. For each metabolite T-values were obtained for the reference and control treatment and compared by the Welch test to asses whether these two groups are significantly different. The maximum absolute value of the respective TVALUE was taken to indicate the most important metabolite for the pattern.

The changes of the group of plasma metabolites being indicative for kidney toxicity after treatment of the rats are shown in the following tables:

TABLE 1a
Markers for kidney toxicity (diuretic effect) in female rats; Significant
up-regulation changes (p-Value ≦ 0.2) are marked (*). For some
metabolites (marked with #), additional information are provide
in Table 10. All compounds were administered in high dose.

| | Theophylline | Caffeine | Furosemide |
|---|---|---|---|
| Metabolite | f28 | f28 | f28 |
| Valine | 1.29 * | 1.15 * | 1.17 * |
| trans-4-Hydroxyproline | 1.91 * | 1.23 * | 1.16 * |
| Proline | 1.57 | 1.23 | 1.16 * |
| Glycine | 1.32 * | 1.5 * | 1.14 * |
| Pantothenic acid | 1.72 * | 1.23 * | 1.15 * |
| Histidine | 1.25 * | 1.29 * | 1.35 * |
| Threonic acid | 1.14 | 1.46 * | 1.39 * |

TABLE 1b
Markers for kidney toxicity (diuretic effect) in female rats; Significant
down-regulation changes (p-Value ≦ 0.2) are marked (*). For
some metabolites (marked with #), additional information are
provide in Table 10. All compounds were administered in high dose.

| | Theophylline | Caffeine | Furosemide |
|---|---|---|---|
| Metabolite | f28 | f28 | f28 |
| 18-Hydroxy-11- | 0.5 * | 0.38 * | 0.36 * |
| deoxycorticosterone | | | |
| Creatinine | 0.8 * | 0.84 * | 0.77 * |
| Hippuric acid | 0.28 * | 0.38 * | 0.47 * |

TABLE 1c
Markers for kidney toxicity (diuretic effect) in male rats; Significant up-regulation changes (p-Value ≦ 0.2) are marked (*).
For some metabolites (marked with #), additional information are provide in Table 10. All compounds were administered in high dose.

| | Theobromine | | | Theophylline | | | Caffeine | | | Lisinopril | | |
|---|---|---|---|---|---|---|---|---|---|---|---|---|
| Metabolite | m7 | m14 | m28 | m7 | m14 | m28 | m7 | m14 | m28 | m7 | m14 | m28 |
| Choline plasmalogen No 02# | 1.38 * | 1.16 | 1.34 * | 1.65 * | 1.12 * | 1 | 1.22* | 1.18* | 1.26 * | 1.07* | 1.27 * | 1.11 * |
| Sphingomyelin (d18:2, C16:0)# | 1.41 * | 1.42 * | 1.9 * | 1.61 * | 1.28 * | 1.17 * | 1.75* | 2.34* | 2.05 * | 1.21 * | 1.38 * | 1.13 * |
| Choline plasmalogen No 03# | 1.08 * | 1.1 | 1.1 * | 1.29 | 1.18 * | 1.03 | 1.55 * | 1.56 * | 1.25 * | 1.12 * | 1.1 * | 1.21 * |
| Threonine | 1.32 * | 1.53 * | 1.42 * | 1.22 * | 1.27 * | 1.44 * | 1.2 * | 1.58 * | 1.59 * | 0.99 | 1.15 | 1.12 * |
| Ceramide (d18:1, C24:1) | 1.21 * | 1.45 * | 1.52 | 0.75 | 1.94 * | 1.17 | 1.2 | 1.7 * | 2.43 * | 1.12 | 1.32 * | 1.36 * |
| Ceramide (d18:1, C24:0) | 0.84 | 1.35 * | 1.33 * | 0.92 | 1.56 * | 0.95 | 1.11 | 2.08 * | 1.82 * | 1.22 * | 1.19 * | 1.24 * |

TABLE 1d
Markers for kidney toxicity (diuretic effect) in male rats; Significant up-regulation changes (p-Value ≦ 0.2) are marked (*).
For some metabolites (marked with #), additional information are provide in Table 10. All compounds were administered in high dose.

| | Theobromine | | | Theophylline | | | Caffeine | | | Lisinopril | | |
|---|---|---|---|---|---|---|---|---|---|---|---|---|
| Metabolite | m7 | m14 | m28 | m7 | m14 | m28 | m7 | m14 | m28 | m7 | m14 | m28 |
| Malate | 1.08 | 1.65 * | 1.11 | 0.76 * | 0.9 | 0.72 * | 0.61 * | 0.55 * | 0.54 * | 0.75 * | 0.6 * | 0.65 * |
| Glutamate | 0.74 * | 0.94 * | 0.87 * | 0.64 * | 1.04 | 0.74 * | 0.55 * | 0.65 * | 0.7 * | 0.94 * | 0.85 * | 0.87 * |
| Lysophosphatidylcholine | 0.8 * | 0.86 * | 0.8 * | 0.71 * | 0.87 * | 0.81 * | 0.85 * | 0.87 * | 0.89 * | 0.97 | 0.94 | 0.9 * |
| (C20:4)# | | | | | | | | | | | | |

TABLE 2a
Markers for kidney toxicity (glomerular-tubular defect) in female rats;
Significant up-regulation changes (p-Value ≦ 0.2) are marked (*).
For some metabolites (marked with #), additional information are
provide in table 10. All compounds were administered in high dose.

| | Penicillamine | | Captopril | | Cyclosporin A | |
|---|---|---|---|---|---|---|
| Metabolite | f14 | f28 | f14 | f28 | f14 | f28 |
| Creatine | 1.23 * | 1.11 * | 1.18* | 1.33 * | 1.37 * | 1.17 |
| Elaidic acid | 1.2 | 1.35 * | 1.18 | 1.22 * | 1.34 * | 0.88 |
| (C18:trans[9]1) | | | | | | |
| 3- | 0.93 | 1.41 * | 1.2 | 1.21 * | 1.43 * | 0.74 |
| Indoxylsulfate | | | | | | |
| 3- | 0.87 | 1.53 * | 1.35 | 1.21 * | 1.91 * | 0.74 |
| Hydroxyindole | | | | | | |
| Xylitol | 1.29 * | 1.13 | 1.17 | 1.31 * | 0.92 | 1.21 * |
| Creatinine | 1.69 * | 1.11 | 1.06 | 1.93 * | 1.8 * | 1.49 * |
| Lysophospha- | 1.14 | 1.34 * | 1.04 | 1.24 * | 1.27 | 1.11 |
| tidylcholine | | | | | | |
| (C20:4)# | | | | | | |

TABLE 2b
Markers for kidney toxicity (glomerular-tubular defect)
in female rats; Significant down-regulation changes (p-
Value ≦ 0.2) are marked (*). For some metabolites
(marked with #), additional information are provide
in table 10. All compounds were administered in high dose.

| | Penicillamine | | Captopril | | Cyclosporin A | |
|---|---|---|---|---|---|---|
| Metabolite | f14 | f28 | f14 | f28 | f14 | f28 |
| Glucosamine | 0.75 | 0.48 * | 0.4 * | 1.06 | 0.73 * | 0.58 * |
| Mannosamine | 0.64 * | 0.5 * | 0.69 * | 1.04 | 0.73 * | 0.63 * |
| Histamine | 1.36 | 0.52 * | 0.93 | 0.81 * | NA | NA |
| Adrenaline | 0.55 * | 1.14 | 1.27 | 0.28 * | NA | NA |
| (Epinephrine) | | | | | | |

TABLE 2c
Markers for kidney toxicity (glomerular-tubular defect) in male rats; Significant up-regulation
changes (p-Value ≦ 0.2) are marked (*). For some metabolites (marked with #), additional
information are provide in table 10. All compounds were administered in high dose.

| | Tricresyl phosphate | | | Captopril | | | Penicillamine | | |
|---|---|---|---|---|---|---|---|---|---|
| Metabolite | m7 | m14 | m28 | m7 | m14 | m28 | m7 | m14 | m28 |
| Threonic acid | 1.32 * | 1.5 * | 1.53 * | 1.37 * | 1.27 * | 1.14 * | 1.47 * | 1.4 * | 1.7 * |
| Lysophosphatidylcholine | 1.19 | 1.27 * | 1.15 * | 1.07 * | 1.2 * | 1.09 | 1.23 * | 1.28 * | 1.1 |
| (C18:2)# | | | | | | | | | |
| Creatine | 1.16 * | 1.42 * | 1.11 * | 1.16 * | 1.06 * | 1.31 | 1.11 * | 1.35 * | 1.27 * |
| Glycerol-3-phosphate, | 0.83 * | 0.89 | 0.89 | 1.28 * | 0.97 | 1.29 * | 1.21 * | 1.42 * | 1.4 * |
| polar fraction | | | | | | | | | |
| Creatinine | 0.77 | 0.74 | 0.78 * | 1.32 | 1.12 * | 1.05 | 1.16 | 1.8 * | 1.82 * |

TABLE 2d
Markers for kidney toxicity (glomerular-tubular defect) in male rats; Significant up-regulation
changes (p-Value ≦ 0.2) are marked (*). For some metabolites (marked with #), additional
information are provide in table 10. All compounds were administered in high dose.

| | Tricresyl phosphate | | | Captopril | | | Penicillamine | | |
|---|---|---|---|---|---|---|---|---|---|
| Metabolite | m7 | m14 | m28 | m7 | m14 | m28 | m7 | m14 | m28 |
| Cysteine | 0.44 * | 0.51 * | 0.55 * | 0.59 * | 0.77 * | 0.72 * | 0.46 * | 0.51 * | 0.85 * |
| Metanephrine | 0.43 * | 0.74 * | 0.76 | 0.81 * | 0.7 * | 0.76 * | 0.83 * | 0.43 * | 0.68 * |
| Normetanephrine | 0.74 * | 0.71 * | 1.03 | 0.9 | 0.89 * | 0.73 * | 0.79 * | 0.66 * | 0.68 * |
| 4-Hydroxy-3- | 0.95 | 0.93 * | 0.83 * | 1.07 | 0.91 * | 0.86 * | 0.91 * | 0.84 * | 0.79 * |
| methoxyphenylglycol | | | | | | | | | |
| (HMPG) | | | | | | | | | |
| Isopalmitic acid (C16:0) | 0.84 * | 0.76 * | 0.62 * | 0.78 * | 0.64 * | 0.83 | 0.75 * | 1.02 | 1.23 |

TABLE 3a
Markers for kidney toxicity (tubular defect) in female rats; Significant
up-regulation changes (p-Value ≦ 0.1) are marked (*). For some
metabolites (marked with #), additional information are provide
in table 10. All compounds were administered in high dose.

| | Beta-ionone | | Carboplatin | | Furosemide | |
|---|---|---|---|---|---|---|
| Metabolite | f14 | f28 | f14 | f28 | f14 | f28 |
| Creatine | NA | NA | 1.22 * | 1.41 * | 1.44 * | 1.56 * |
| Lysophospha- | 1.34 * | 1.42 * | 0.98 | 1.12 * | 1.14 * | 1.16 * |
| tidylcholine | | | | | | |
| (C18:1)# | | | | | | |
| Histidine | 1.31 * | 1.21 * | 1.03 | 1.3 * | 1.1 * | 1.35 * |
| Valine | 1.19 * | 1.42 | 1.07 * | 1.25 * | 1.12 | 1.17 * |
| Leucine | 1.26 | 1.36 | 1.13 * | 1.38 * | 1.26 | 1.34 * |
| Galactose, lipid | 2.02 * | 2.58 * | 1.17 * | 0.84 | 1.42 * | 0.92 |
| fraction | | | | | | |
| Phosphati- | 1.99 * | 1.76 * | 1.07 * | 0.97 | 1.05 * | 0.98 |
| dylcholine | | | | | | |
| No 02# | | | | | | |
| Isoleucine | 1.14 | 1.32 | 1.09 * | 1.35 * | 1.11 | 1.27 * |
| Glycerol-3- | 0.98 | 1.25 | 1.66 * | 2.2 * | 1.51 * | 1.42 * |
| phosphate, | | | | | | |
| polar fraction | | | | | | |
| TAG No 05# | 1.65 * | 1.5 | 1.9 * | 1.27 * | 3.01 * | 1.83 * |
| Glycine | 1.17 | 1.18 | 1.08 | 1.17 * | 1.16 | 1.14 * |
| Threonic acid | 1.11 | 1.21 | 1.14 | 1.23 * | 1.06 * | 1.39 * |
| Xylitol | 1.32 * | 1.88 | 0.83 | 1.56 * | 1.56 * | 1.33 * |
| Uracil | 0.94 | 0.94 | 1.07 | 1.37 * | 1.17 | 1.41 * |
| Glucuronic | 2.55 * | 2.03 * | 1.36 | 1.24 * | 1.41 * | 1.33 * |
| acid | | | | | | |
| Creatinine | 1.61 | 1.61 | 0.85 | 1.59 * | 1.53 * | 2.2 * |
| Threonine | 1.57 * | 1.46 * | 0.99 | 1.24 * | 1.18 * | 1.07 |

TABLE 3b
Markers for kidney toxicity (tubular defect) in female rats; Significant
down-regulation changes (p-Value ≦ 0.1) are marked (*). For
some metabolites (marked with #), additional information are
provide in table 10. All compounds were administered in high dose.

| | Beta-ionone | | Carboplatin | | Furosemide | |
|---|---|---|---|---|---|---|
| Metabolite | f14 | f28 | f14 | f28 | f14 | f28 |
| Indole-3-acetic | 0.62 * | 0.81 | 0.38 * | 0.65 | 0.4 * | 0.7 * |
| acid | | | | | | |
| Glycerol, polar | 0.83 | 0.65 | 0.88 | 0.78 * | 0.9 | 0.79 * |
| fraction | | | | | | |
| Sucrose | 1.02 | 1.37 | 0.67 * | 0.61 | 0.49 * | 0.52 |
| Hippuric acid | 0.55 | 0.5 * | 0.59 | 0.3 * | 0.55 | 0.47 * |
| Cysteine | 0.86 | 1.06 | 0.83 * | 0.67 | 0.69 * | 0.94 |
| Spermidine | 0.54 * | 1.17 | 0.95 | 0.69 * | 0.77 * | 0.75 |

TABLE 3c
Markers for kidney toxicity (tubular defect) in male rats; Significant
up-regulation changes (p-Value ≦ 0.1) are marked (*). For some
metabolites (marked with #), additional information are provide
in table 10. All compounds were administered in high dose.

| | Carboplatin | | Furosemide | |
|---|---|---|---|---|
| Metabolite | m14 | m28 | m14 | m28 |
| Pseudouridine | 1.33 * | 1.11 * | 1.56 * | 1.49 * |
| Taurine | 1.18 * | 1.54 * | 1.69 * | 1.67 * |
| Allantoin | 1.4 | 1.24 * | 1.9 * | 1.65 * |
| Kynurenic acid | 1.21 * | 1.7 | 2.01 * | 1.44 |
| Glycerol-3-phosphate, | 1.59 * | 1.61 * | 1.35 * | 1.88 * |
| polar fraction | | | | |
| Creatine | 1.32 * | 1.42 * | 1.62 * | 1.22 |
| Nervonic acid | 1.02 | 1.51 * | 2.97 * | 1.46 * |
| (C24:cis[15]1) | | | | |
| Urea | 1.47 * | 1.08 | 2.21 * | 1.73 * |
| Pantothenic acid | 1.17 | 1.47 * | 1.19 * | 1.58 * |
| Histidine | 1.02 | 1.22 * | 1 | 1.21 * |

TABLE 3d
Markers for kidney toxicity (tubular defect) in male rats; Significant
up-regulation changes (p-Value ≦ 0.1) are marked (*). For some
metabolites (marked with #), additional information are provide
in table 10. All compounds were administered in high dose.

| | Carboplatin | | Furosemide | |
|---|---|---|---|---|
| Metabolite | m14 | m28 | m14 | m28 |
| TAG (C18:1, C18:2)# | 0.79 * | 0.62 * | 0.79 | 0.7 * |
| 16-Methylheptadecanoic acid | 0.74 * | 0.51 * | 0.61 * | 0.72 * |
| Linolenic acid | 0.75 | 0.7 * | 0.85 | 0.68 * |
| (C18:cis[9,12,15]3) | | | | |
| Phosphatidylcholine No 04# | 0.74 * | 0.78 | 0.59 * | 0.68 * |
| Phosphatidylcholine No 02# | 0.87 * | 0.91 * | 1.01 | 0.88 * |

TABLE 4a
Markers for kidney toxicity (inhibition of weak acid excretion) in female rats; Significant up-regulation changes (p-Value ≦ 0.2) are
marked (*). For some metabolites (marked with #), additional information are provide in table 10. All compounds were administered in high dose.

| | Dichlorprop-p | | | MCPA | | | Mecoprop-p | | | Pentachlorophenol | | |
|---|---|---|---|---|---|---|---|---|---|---|---|---|
| Metabolite | f7 | f14 | f28 | f7 | f14 | f28 | f7 | f14 | f28 | f7 | f14 | f28 |
| Lysophosphatidylcholine | 1.47 * | 1.44 * | 1.16 * | 1.38 | 1.45 * | 1.35 * | 1.26 * | 1.67 * | 1.74 * | 1.32 * | 1.41 * | 1.66 * |
| (C18:2)# | | | | | | | | | | | | |
| gamma-Linolenic acid | 6.14 * | 6.64 * | 3.31 * | 4.23 * | 2.93 * | 2.99 * | 3.03 * | 3.62 * | 4.04 * | 0.78 * | 1.81 | 3.23 * |
| (C18:cis[6,9,12]3) | | | | | | | | | | | | |
| Phosphatidylcholine | 1.72 * | 1.25 * | 1.08 * | 2.03 * | 2.09 * | 1.61 * | 1.47 | 1.74 * | 1.47 * | 1.05 * | 1.57 * | 1.67 * |
| (C16:0, C20:5)# | | | | | | | | | | | | |
| Phosphatidylcholine | 2.98 * | 2.37 * | 1.81 * | 2.92 * | 2.88 * | 2.45 * | 1.06 | 2.03 * | 2.28 | 0.87 | 1.32 * | 1.54 * |
| (C16:1, C18:2)# | | | | | | | | | | | | |
| Glycerol, lipid | 3.12 * | 2.68 * | 2.19 * | 1.69 * | 1.38 * | 1.6 * | 1.96 * | 2.17 * | 2.15 * | 1.56 * | 2.38 * | 1.61 * |
| fraction# | | | | | | | | | | | | |
| Phosphatidylcholine | 1.36 * | 1.43 * | 1.44 * | 1.57 * | 1.74 * | 1.56 * | 1.23 | 1.33 * | 1.4 * | 0.98 | 1.54 * | 1.41 * |
| (C18:0, C18:1)# | | | | | | | | | | | | |
| Linoleic acid | 2.28 * | 2.34 * | 1.14 | 1.66 * | 1.36 * | 1.28 * | 1.51 * | 1.86 * | 1.51 * | 1.35 * | 1.8 * | 1.73 * |
| (C18:cis[9,12]2) | | | | | | | | | | | | |
| Phosphatidylcholine | 1.88 * | 2.33 * | 1.71 * | 1.68 | 2.12 * | 1.78 * | 1.65 | 1.9 * | 1.78 * | 0.85 | 1.4 * | 1.3 * |
| (C18:1, C18:2)# | | | | | | | | | | | | |
| Phosphatidylcholine | 1.47 * | 1.51 * | 1.29 * | 1.66 * | 1.82 * | 1.45 * | 1.25 * | 1.58 * | 1.34 * | 1.02 | 1.34 * | 1.49 * |
| (C16:0, C18:2) | | | | | | | | | | | | |
| Phosphatidylcholine | 1.43 * | 1.14 | 1.26 * | 1.52 * | 1.8 * | 1.78 * | 1.17 | 1.44 * | 1.4 * | 1.04 | 1.25 * | 1.1 * |
| (C16:0, C16:0)# | | | | | | | | | | | | |

TABLE 4b
Markers for kidney toxicity (inhibition of weak acid excretion) in female rats; Significant down-
regulation changes (p-Value ≦ 0.2) are marked (*). For some metabolites (marked with #), additional
information are provide in table 10. All compounds were administered in high dose.

| | Dichlorprop-p | | | MCPA | | | Mecoprop-p | | | Pentachlorophenol | | |
|---|---|---|---|---|---|---|---|---|---|---|---|---|
| Metabolite | f7 | f14 | f28 | f7 | f14 | f28 | f7 | f14 | f28 | f7 | f14 | f28 |
| Proline | 0.85 * | 0.79 * | 0.72 * | 0.61 * | 0.45 * | 0.42 * | 0.7 * | 0.72 * | 0.66 * | 0.73 * | 0.74 * | 0.74 * |
| Indole-3-lactic | 0.16 * | 0.22 * | 0.32 * | 0.24 * | 0.24 * | 0.23 * | 0.6 * | 0.3 * | 0.43 * | 0.62 * | 0.65 * | 0.49 * |
| acid | | | | | | | | | | | | |
| trans-4- | 0.88 | 0.89 * | 0.77 * | 0.57 | 0.47 * | 0.6 * | 0.6 * | 0.7 * | 0.77 * | 0.87 * | 0.92 | 0.94 * |
| Hydroxyproline | | | | | | | | | | | | |
| Ketoleucine | 0.5 * | 0.55 * | 0.5 * | 0.42 * | 0.21 * | 0.15 * | 0.6 * | 0.71 * | 0.71 * | 0.6 * | 0.64 * | 0.6 * |
| Tryptophan | 0.17 * | 0.2 * | 0.31 * | 0.17 * | 0.18 * | 0.16 * | 0.5 * | 0.36 * | 0.44 * | 0.6 * | 0.62 * | 0.54 * |
| Arginine | 0.9 * | 0.91 | 0.79 | 0.62 * | 0.58 * | 0.58 * | 0.82 * | 0.78 * | 0.78 * | 0.56 * | 0.72 | 0.44 * |

TABLE 4c
Markers for kidney toxicity (inhibition of weak acid excretion) in male rats; Significant up-regulation changes (p-Value ≦ 0.2) are
marked (*). For some metabolites (marked with #), additional information are provide in table 10. All compounds were administered in high dose.

| | Dichlorprop-p | | | MCPA | | | Probenecid | | | Mecopnop-p | | |
|---|---|---|---|---|---|---|---|---|---|---|---|---|
| Metabolite | m7 | m14 | m28 | m7 | m14 | m28 | m7 | m14 | m28 | m7 | m14 | m28 |
| Phosphatidylcholine | 1.48 * | 1.51 * | 1.19 * | 1.73 * | 1.82 * | 2.11 * | 1.28 * | 1.36 * | 1.15 * | 1.43 * | 1.2 * | 1.22 * |
| (C16:0, C20:5)# | | | | | | | | | | | | |
| gamma-Linolenic acid | 2.07 | 1.93 * | 1.13 | 1.69 * | 2.68 * | 2.54 * | 2.14 * | 2.74 * | 2.26 * | 1.08 | 1.21 | 1.08 |
| (C18:cis[6,9,12]3) | | | | | | | | | | | | |
| Ceramide(d18:1, C24:0) | 1.01 | 1.54 * | 1.07 | 1.2 | 2.39 * | 1.72 * | 1.23 | 1.53 * | 1.27 * | 1.09 | 1.46 * | 1.19 * |
| Cholesterolester No 01# | 2.12 * | 2.21 * | 1.05 * | 0.97 | 1.27 * | 1.32 * | 1.15 * | 1.01 | 1 | 1.41 | 1.32 * | 1.25 * |
| Lysophosphatidylethanolamine | 1.03 | 1.22 * | 1 | 1.16 | 1.08 | 1.1 | 1.17 * | 1.7 * | 1.17 * | 1.17 | 1.11 * | 1.24 * |
| (C22:5)# | | | | | | | | | | | | |
| 3-Indoxylsulfate | 5.58 * | 3.28 * | 3.25 * | 0.72 | 1.53 * | 1.92 * | 3.55 * | 4.18 * | 6.58 * | 4.14 * | 2.1 * | 3 * |
| Aspartate | 1.27 * | 1.18 * | 2.27 * | 0.58 * | 1.01 | 0.69 | 0.71 | 0.83 | 1.16 | 1.23 * | 0.93 | 0.77 |

TABLE 4d
Markers for kidney toxicity (inhibition of weak acid excretion) in male rats; Significant down-regulation changes (p-Value ≦ 0.2) are
marked (*). For some metabolites (marked with #), additional information are provide in table 10. All compounds were administered in high dose.

| | Dichlorprop-p | | | MCPA | | | Probenecid | | | Mecoprop-p | | |
|---|---|---|---|---|---|---|---|---|---|---|---|---|
| Metabolite | m7 | m14 | m28 | m7 | m14 | m28 | m7 | m14 | m28 | m7 | m14 | m28 |
| Lysophosphatidylcholine | 0.77 * | 0.78 * | 0.83 * | 0.81 * | 0.83 * | 0.73 * | 0.94 * | 0.95 * | 0.98 | 0.75 * | 0.78 * | 0.77 * |
| (C18:0)# | | | | | | | | | | | | |
| Methionine | 0.77 * | 0.73 * | 0.82 | 0.66 * | 0.59 * | 0.64 * | 0.89 * | 0.8 * | 0.97 | 0.76 * | 0.82 * | 0.8 * |
| Indole-3-lactic acid | 0.25 * | 0.29 * | 0.52 * | 0.36 * | 0.38 | 0.3 * | 0.69 * | 0.58 * | 0.65 * | 0.51 * | 0.5 * | 0.55 * |
| Nervonic acid | 0.62 * | 0.41 * | 0.66 * | 0.67 * | 0.74 * | 0.76 * | 0.52 * | 0.93 | 0.82 * | 0.53 * | 0.6 * | 0.53 * |
| (C24:cis[15]1) | | | | | | | | | | | | |
| Choline plasmalogen | 0.42 * | 0.57 * | 0.65 * | 0.64 * | 0.76 * | 0.45 * | 0.79 * | 1 | 0.93 | 0.53 * | 0.63 * | 0.59 * |
| (C18, C20:4) | | | | | | | | | | | | |
| Phosphatidylcholine | 0.36 * | 0.4 * | 0.51 * | 0.36 * | 0.55 * | 0.24 * | 0.99 | 0.98 * | 0.98 * | 0.32 * | 0.41 * | 0.38 * |
| (C18:0, C20:4)# | | | | | | | | | | | | |
| Cytosine | 0.44 * | 0.62 * | 0.69 | 0.63 * | 0.63 * | 0.6 * | 0.7 * | 0.69 * | 0.68 * | 0.72 * | 0.73 * | 0.66 * |
| Ribal | 0.5 * | 0.78 * | 1.08 | 0.94 * | 0.89 * | 0.77 * | 0.78 * | 0.87 * | 0.68 * | 0.76 * | 0.82 * | 0.7 * |
| Arachidonic acid | 0.2 * | 0.29 * | 0.41 * | 0.27 * | 0.42 * | 0.26 * | 0.84 * | 0.96 | 0.94 * | 0.28 * | 0.34 * | 0.26 * |
| (C20:cis[5,8,11,14]4) | | | | | | | | | | | | |
| Lysine | 0.44 * | 0.52 * | 0.51 * | 0.4 * | 0.3 * | 0.33 * | 0.94 * | 0.79 * | 0.97 | 0.59 * | 0.6 * | 0.5 * |
| Aspartate | 1.27 | 1.18 | 2.27 | 0.58 * | 1.01 | 0.69 * | 0.71 * | 0.83 | 1.16 | 1.23 | 0.93 | 0.77 |
| Arginine | 0.74 * | 0.65 * | 0.85 * | 0.72 * | 0.57 * | 0.68 * | 0.86 * | 0.79 | 0.91 | 0.79 * | 0.7 * | 0.78 * |
| Glucose | 0.98 | 0.92 * | 0.91 | 0.75 * | 0.72 * | 0.6 * | 1.11 | 0.8 * | 0.77 * | 0.9 | 1.15 | 0.8 * |

TABLE 5a
Markers for kidney toxicity (tubular necrosis) in female rats; Significant
up-regulation changes (p-Value ≦ 0.2) are marked (*). For some
metabolites (marked with #), additional information are provide
in table 10. All compounds were administered in high dose.

| | Amphotericin | Tobramycin | Hexachloro- |
|---|---|---|---|
| | B | s.c. | butadiene |
| Metabolite | f28 | f28 | f28 |
| Docosahexaenoic acid | 1.32 * | 1.05 * | 1.19 * |
| (C22:cis[4,7,10,13,16,19]6) | | | |
| Leucine | 1.21 * | 1.26 * | 1.34 * |
| Docosapentaenoic acid | 1.23 * | 1.56 * | 1.61 * |
| (C22:cis[7,10,13,16,19]5) | | | |
| Phosphate (inorganic | 1.25 * | 1.3 * | 1.19 * |
| and organic phosphates) | | | |
| Glycerol, lipid fraction | 1.31 * | 1.2 * | 1.29 * |
| Eicosanoic acid (C20:0) | 1.18 * | 1.05 | 1.56 * |

TABLE 5b
Markers for kidney toxicity (tubular necrosis) in female rats; Significant
down-regulation changes (p-Value ≦ 0.2) are marked (*). For some
metabolites (marked with #), additional information are provide
in table 10. All compounds were administered in high dose.

| | Amphotericin | Tobramycin | Hexachloro- |
|---|---|---|---|
| | B | s.c. | butadiene |
| Metabolite | f28 | f28 | f28 |
| Hippuric acid | 0.44 * | 0.14 * | 0.35 * |
| Lactate | 0.68 * | 0.64 * | 0.62 * |

TABLE 6a
Markers for kidney toxicity (ACE inhibitor induced like
toxicity) in male rats; Significant up-regulation changes
(p-Value ≦ 0.2) are marked (*). For some metabolites
(marked with #), additional information are provide
in table 10. All compounds were administered in high dose.

| | Ramipril | | | Lisinopril | | |
|---|---|---|---|---|---|---|
| Metabolite | m7 | m14 | m28 | m7 | m14 | m28 |
| Lysine | 1.37 * | 1.21 * | 1.17 * | 0.92 | 1.13 * | 1.23 * |
| Glycine | 1.24 * | 1.5 * | 1.37 * | 1.21 * | 1.25 * | 1.29 * |
| 1,5-Anhydro- | 1.02 | 1.21 * | 1.17 * | 1.22 * | 1.2 * | 1.53 * |
| sorbitol | | | | | | |

TABLE 6b
Markers for kidney toxicity (ACE inhibitor induced like
toxicity) in male rats; Significant down-regulation changes
(p-Value ≦ 0.2) are marked (*). For some metabolites
(marked with #), additional information are provide
in table 10. All compounds were administered in high dose.

| | Ramipril | | | Lisinopril | | |
|---|---|---|---|---|---|---|
| Metabolite | m7 | m14 | m28 | m7 | m14 | m28 |
| Cytosine | 0.7 * | 0.85 * | 0.91 | 0.97 | 0.89 * | 1 |
| Glutamate | 0.92 * | 0.71 * | 0.61 * | 0.94 * | 0.85 * | 0.87 * |

TABLE 7a
Markers for kidney toxicity (interstitial nephritis) in male rats; Significant up-regulation
changes (p-Value ≦ 0.2) are marked (*). For some metabolites (marked with #), additional
information are provided in Table 10. All compounds were administered in high dose.

| | Lithocholic acid | | | Dipyrone | | | Ethyl benzene | | |
|---|---|---|---|---|---|---|---|---|---|
| Metabolite | m7 | m14 | m28 | m7 | m14 | m28 | m7 | m14 | m28 |
| Lignoceric acid | 1.31 * | 1.37 * | 1.3 * | 1.02 * | 1.08 | 1.14 * | 1.31 * | 1.55 * | 1.23 * |
| (C24:0) | | | | | | | | | |
| Creatine | 1.29 * | 1.27 * | 1.54 * | 1.45 * | 1.48 * | 1.07 | 1.4 * | 1.35 * | 1.34 * |
| Serine | 1.18 * | 1.16 * | 1.51 * | 1.22 * | 1.38 * | 1.67 * | 1.12 * | 1.5 * | 1.54 * |
| Threonine | 1.37 * | 1.13 * | 1.55 * | 1.09 * | 1.78 * | 1.88 * | 1.31 * | 1.72 * | 2.44 * |
| Eicosanoic acid | 1.81 * | 1.12 * | 1.58 * | 1.78 * | 1.27 | 0.97 | 1.41 * | 1.3 * | 1.76 * |
| (C20:0) | | | | | | | | | |
| Phosphatidylcholine | 1.05 * | 1.14 * | 1.15 * | 1.01 | 1.08 * | 1.09 * | 1.05 * | 1.06 * | 1.05 |
| (C18:2, C20:4)# | | | | | | | | | |
| myo-Inositol-2- | 1.06 | 1.01 | 1.51 * | 1.16 * | 1.26 * | 1.6 * | 1.39 * | 0.88 | 1.3 * |
| phosphate | | | | | | | | | |
| Glucuronic acid | 0.85 * | 1 | 1 | 1.72 * | 1.56 * | 1.69 * | 1.08 | 1.17 * | 1.26 * |
| Sphingomyelin | 1.27 * | 1.22 * | 1.09 | 1.32 * | 1.19 * | 1.03 | 1.69 * | 1.5 * | 1.48 * |
| (d18:1, C16:0)# | | | | | | | | | |
| Creatinine | 1.12 | 1.21 * | 1.27 * | 1.63 * | 1.71 * | 1.15 | 1.41 * | 1.58 * | 1.33 * |

TABLE 7b
Markers for kidney toxicity (interstitial nephritis) in male rats; Significant down-regulation
changes (p-Value ≦ 0.2) are marked (*). For some metabolites (marked with #), additional
information are provided in Table 10. All compounds were administered in high dose.

| | Lithocholic acid | | | Dipyrone | | | Ethyl benzene | | |
|---|---|---|---|---|---|---|---|---|---|
| Metabolite | m7 | m14 | m28 | m7 | m14 | m28 | m7 | m14 | m28 |
| Kynurenic acid | 0.75 * | 0.75 * | 0.71 | 0.72 * | 0.82 | 0.78 | 0.71 * | 0.83 * | 0.8 * |
| Isopalmitic acid | 0.63 * | 0.54 * | 0.68 * | 0.73 | 0.69 * | 0.64 * | 0.55 * | 0.57 * | 0.59 * |
| (C16:0) | | | | | | | | | |
| Glycerol, polar | 0.93 | 0.86 | 0.73 * | 0.87 * | 1.2 | 0.74 * | 0.98 | 0.87 | 0.75 * |
| fraction | | | | | | | | | |

TABLE 8a
Markers for kidney toxicity (direct tubular effect) in male rats;
Significant up-regulation changes (p-Value ≦ 0.2) are marked
(*). For some metabolites (marked with #), additional information
are provide in table 10. All compounds were administered in high dose.

| | Hexachloro- | |
|---|---|---|
| | butadiene | Hydroquinone |
| Metabolite | m28 | m28 |
| Lysophosphatidylethanolamine (C22:0) | 1.33 * | 1.26 * |
| Leucine | 1.17 * | 1.23 * |
| TAG No 02# | 1.47 * | 1.91 * |
| Glycerol-3-phosphate, polar fraction | 1.58 * | 1.64 * |
| Ornithine | 1.35 * | 1.24 * |
| Threonine | 1.3 * | 1.03 |
| Isoleucine | 1.11 * | 1.2 * |

TABLE 8b
Markers for kidney toxicity (direct tubular effect) in male rats;
Significant down-regulation changes (p-Value ≦ 0.2) are marked
(*). For some metabolites (marked with #), additional information
are provide in table 10. All compounds were administered in high dose.

| | Hexachloro- | |
|---|---|---|
| | butadiene | Hydroquinone |
| Metabolite | m28 | m28 |
| Oleic acid (C18:cis[9]1) | 0.71 * | 0.75 * |
| TAG (C16:0, C16:1)# | 0.51 * | 0.4 * |
| DAG (C18:1, C18:2)# | 0.66 * | 0.43 * |
| trans-4-Hydroxyproline | 0.74 * | 0.87 * |
| 18-Hydroxy-11-deoxycorticosterone | 0.49 * | 0.23 * |
| Citrate | 0.93 * | 0.82 * |
| 14-Methylhexadecanoic acid | 0.82 * | 0.81 |
| Linolenic acid (C18:cis[9,12,15]3) | 0.6 * | 0.56 * |
| Palmitoleic acid (C16:cis[9]1) | 0.25 * | 0.76 * |
| Isopalmitic acid (C16:0) | 0.61 * | 0.62 |
| Ketoleucine | 0.91 * | 0.75 * |
| 16-Methylheptadecanoic acid | 0.51 * | 0.76 |
| Elaidic acid (C18:trans[9]1) | 0.8 * | 0.84 |
| Lysophosphatidylcholine (C18:0)# | 0.97 * | 0.99 * |

TABLE 9
Compounds and dosing (CMC = Carboxymethyl cellulose)

| | | | Dosage | |
|---|---|---|---|---|
| Compound | Synonym | CAS no | administered | Details |
| Amphotericin B | na | 1397-89-3 | 3 mg/kg | in 0.9% NaCl; |
| | | | body weight | administration |
| | | | intraperitoneal | volume: 5 ml/kg |
| | | | | bw |
| Beta-ionone | na | 79-77-6 | 10,000 ppm | mixture in |
| | | | in the diet | the diet |
| Caffeine | 3,7-Dihydro-1,3,7- | 58-08-2 | 5,000 ppm | mixture in |
| | trimethyl-1H-purine- | | in the diet | the diet |
| | 2,6-dione | | | |
| Captopril | N-[(S)-3-Mercapto-2- | 62571-86-2 | 200 mg/kg | In drinking water; |
| | methylpropionyl]-L- | | body weight | administration |
| | proline | | by gavage | volume: 10 ml/kg |
| | | | | bw |
| Carboplatin | cis-Diammine(1,1- | 41575-94-4 | 10 mg/kg | In 0.9% NaCl; |
| | cyclobutanedi- | | body weight | administration |
| | carboxylato) platinum | | i.p., twice | volume: 3 ml/kg |
| | | | a week | bw |
| Cyclosporin A | na | 59865-13-3 | 45 mg/kg | in corn oil, |
| | | | body weight | administration |
| | | | by gavage | volume: 5 ml/kg |
| | | | | body weight |
| Dichlorprop-p | R-2-(2,4- | 15165-67-0 | 2,500 ppm | mixture in |
| | dichlorphenoxy)- | | in the diet | the diet |
| | propanoic acid | | | |
| Dipyrone | Metamizole | 68-89-3 | 900 mg/kg | in corn oil; |
| | | | body weight | administration |
| | | | by gavage | volume: 5 ml/kg |
| | | | | bw. |
| Ethylbenzene | na | 100-41-4 | 750 mg/kg | in corn oil; |
| | | | body weight | administration |
| | | | by gavage | volume: 5 ml/kg |
| | | | | bw |
| Furosemide | 4-Chloro-N-furfuryl-5- | 54-31-9 | 7500 ppm | mixture in the |
| | sulfamoylanthranilic | | in the diet | diet |
| | acid | | (days 0 to 6); | |
| | | | 3000 ppm | |
| | | | in the diet | |
| | | | (from day 7 on) | |
| Hexachlorobutadiene | Perchlorobutadiene | 87-68-3 | 10 mg/kg | in corn oil; |
| | | | body weight | administration |
| | | | by gavage | volume: 5 ml/kg |
| | | | | bw |
| Hydroquinone | 1,4-Dihydroxybenzene | 123-31-9 | 180 mg/kg | In drinking water; |
| | | | bw body weight | administration |
| | | | by gavage | volume: 10 ml/kg |
| | | | (days 0 to 7); | bw. |
| | | | 90 mg/kg | |
| | | | body weight | |
| | | | by gavage | |
| | | | (from day 8 on) | |
| Lisinopril | na | 83915-83-7 | 400 mg/kg | in drinking water |
| | | | body weight | containing 0.5% CMC |
| | | | by gavage | (Tylose CB30000), |
| | | | | administration |
| | | | | volume: 10 ml/kg |
| | | | | bw |
| Lithocholic acid | na | 434-13-9 | 1000 mg/kg | in corn oil; |
| | | | body weight | administration |
| | | | by gavage | volume: 5 ml/kg |
| | | | | bw |
| MCPA | 4-Chloro-2- | 94-74-6 | 2,500 ppm | mixture in |
| | methylphenoxyacetic | | in the diet | the diet |
| | Acid | | | |
| Mecoprop-p | (+)-(R)-2-(4-Chloro-2- | 16484-77-8 | 2,500 ppm | mixture in |
| | methylphenoxy)pro- | | in the diet | the diet |
| | panoic acid | | | |
| Penicillamine | D-(−)-Penicillamine | 52-67-5 | 250 mg/kg | in drinking water; |
| | | | bw | administration |
| | | | by gavage | volume: 10 ml/kg |
| | | | | bw |
| Pentachlorophenol | na | 87-86-5 | 2,500 ppm | mixture in |
| | | | in the diet | the diet |
| | | | (days 0 to 6), | |
| | | | 0 ppm | |
| | | | in the diet | |
| | | | (days 7 to 9), | |
| | | | 1,500 ppm | |
| | | | in the diet | |
| | | | (from day 10 on) | |
| Probenecid | na | 57-66-9 | 800 mg/kg | in corn oil, |
| | | | body weight | administration |
| | | | by gavage | volume: 5 ml/kg |
| | | | | bw |
| Ramipril | na | 87333-19-5 | 10,000 ppm | mixture in |
| | | | in the diet | the diet |
| Theobromine | 2,6-Dihydroxy-3,7- | 83-67-0 | 6000 ppm | mixture in |
| | dimethylpurine | | in the diet | the diet |
| Theophylline | 1,3-dimethyl-3,7- | 58-55-9 | 8,000 ppm | mixture in |
| | dihydro-1H-purine-2,6- | | in the diet | the diet |
| | dione | | (days 0 to 6), | |
| | | | 2,000 ppm | |
| | | | in the diet | |
| | | | (from day 7 on) | |
| Tobramycin s.c. | na | 32986-56-4 | 40 mg/kg | in 0.9% NaCl; |
| | | | body weight | administration |
| | | | subcutaneous, | volume: 1 ml/kg |
| | | | twice daily | bw, twice daily |
| | | | (days 0 to 14), | |
| | | | 20 mg/kg | |
| | | | body weight | |
| | | | subcutaneous, | |
| | | | twice daily | |
| | | | (from day 15 on) | |
| Tricresyl phosphate | Tritolyl phosphate | 1330-78-5 | 500 mg/kg | in corn oil; |
| | | | body weight | administration |
| | | | by gavage | volume: 5 ml/kg |
| | | | | bw |

TABLE 10
Chemical/physical properties of selected biomarkers. These biomarkers
are characterized herein by chemical and physical properties.

| Metabolite | Fragmentation pattern (GC-MS) and description |
|---|---|
| 3-O-Methylsphingosine (d18:1) | 3-O-Methylsphingosine (d18:1) exhibits the following |
| | characteristic ionic fragments when detected with |
| | GC/MS, applying electron impact (EI) ionization |
| | mass spectrometry, after acidic methanolysis and |
| | derivatisation with 2% O-methylhydroxylamine- |
| | hydrochlorid in pyridine and subsequently with N- |
| | methyl-N-trimethylsilyltrifluoracetamid: MS (EI, 70 |
| | eV): m/z (%): 204 (100), 73 (18), 205 (16), 206 (7), |
| | 354 (4), 442 (1). |
| 5-O-Methylsphingosine (d18:1) | 5-O-Methylsphingosine (d18:1) exhibits the following |
| | characteristic ionic fragments when detected with |
| | GC/MS, applying electron impact (EI) ionization |
| | mass spectrometry, after acidic methanolysis and |
| | derivatisation with 2% O-methylhydroxylamine- |
| | hydrochlorid in pyridine and subsequently with N- |
| | methyl-N-trimethylsilyltrifluoracetamid: MS (EI, 70 |
| | eV): m/z (%): 250 (100), 73 (34), 251 (19), 354 (14), |
| | 355 (4), 442 (1). |
| Cholesterolester No 01 | Metabolite belongs to the class of cholesterolesters. |
| | It exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 369.2 (+/−0.5). |
| Choline plasmalogen No 01 | Metabolite belongs to the class of choline |
| | plasmalogens. It exhibits the following characteristic |
| | ionic species when detected with LC/MS, applying |
| | electro-spray ionization (ESI) mass spectrometry: |
| | mass-to-charge ratio (m/z) of the positively charged |
| | ionic species is 772.6 (+/−0.5). |
| Choline plasmalogen No 02 | Metabolite belongs to the class of choline |
| | plasmalogens. It exhibits the following characteristic |
| | ionic species when detected with LC/MS, applying |
| | electro-spray ionization (ESI) mass spectrometry: |
| | mass-to-charge ratio (m/z) of the positively charged |
| | ionic species is 767 (+/−0.5). |
| Choline plasmalogen No 03 | Metabolite belongs to the class of choline |
| | plasmalogens. It exhibits the following characteristic |
| | ionic species when detected with LC/MS, applying |
| | electro-spray ionization (ESI) mass spectrometry: |
| | mass-to-charge ratio (m/z) of the positively charged |
| | ionic species is 768.8 (+/−0.5). |
| DAG (C18:1, C18:2) | DAG (C18:1, C18:2) represents the sum parameter of |
| | diacylglycerols containing the combination of a C18:1 |
| | fatty acid unit and a C18:2 fatty acid unit. The mass- |
| | to-charge ratio (m/z) of the ionised species is 641.6 |
| | Da (+/−0.5 Da). |
| Eicosaenoic acid (C20:1) No | Eicosaenoic acid (C20:1) exhibits the following |
| 02 | characteristic ionic fragments when detected with |
| | GC/MS, applying electron impact (EI) ionization |
| | mass spectrometry, after acidic methanolysis and |
| | derivatisation with 2% O-methylhydroxylamine- |
| | hydrochlorid in pyridine and subsequently with N- |
| | methyl-N-trimethylsilyltrifluoracetamid: MS (EI, 70 |
| | eV): m/z (%): 55 (100), 69 (75), 41 (57), 83 (54), 74 |
| | (53), 97 (45), 110 (20), 292 (13), 293 (13), 124 (12), |
| | 250 (9), 152 (8), 138 (8), 208 (7), 324 (2). |
| Glycerol phosphate, lipid | Glycerol phosphate, lipid fraction represents the sum |
| fraction | parameter of metabolites containing a glycerol-2- |
| | phosphate or a glycerol-3-phosphate moiety and |
| | being present in the lipid fraction after extraction and |
| | separation of the extract into a polar and a lipid |
| | fraction. |
| Lysophosphatidylcholine | Lysophosphatidylcholine (C17:0) represents the sum |
| (C17:0) | parameter of lysoglycerophosphorylcholines |
| | containing a C17:0 fatty acid unit. If detected with |
| | LC/MS, applying electro-spray ionization (ESI) mass |
| | spectrometry, the mass-to-charge ratio (m/z) of the |
| | positively charged ionic species is 510.4 Da (+/−0.5 |
| | Da). |
| Lysophosphatidylcholine | Lysophosphatidylcholine (C18:0) represents the sum |
| (C18:0) | parameter of lysoglycerophosphorylcholines |
| | containing a C18:0 fatty acid unit. If detected with |
| | LC/MS, applying electro-spray ionization (ESI) mass |
| | spectrometry, the mass-to-charge ratio (m/z) of the |
| | positively charged ionic species is 546.6 Da (+/−0.5 |
| | Da). |
| Lysophosphatidylcholine | Lysophosphatidylcholine (C18:1) represents the sum |
| (C18:1) | parameter of lysoglycerophosphorylcholines |
| | containing a C18:1 fatty acid unit. If detected with |
| | LC/MS, applying electro-spray ionization (ESI) mass |
| | spectrometry, the mass-to-charge ratio (m/z) of the |
| | positively charged ionic species is 522.2 Da (+/−0.5 |
| | Da). |
| Lysophosphatidylcholine | Lysophosphatidylcholine (C18:2) represents the sum |
| (C18:2) | parameter of lysoglycerophosphorylcholines |
| | containing a C18:2 fatty acid unit. If detected with |
| | LC/MS, applying electro-spray ionization (ESI) mass |
| | spectrometry, the mass-to-charge ratio (m/z) of the |
| | positively charged ionic species is 542.4 Da (+/−0.5 |
| | Da). |
| Lysophosphatidylcholine | Lysophosphatidylcholine (C20:4) represents the sum |
| (C20:4) | parameter of lysoglycerophosphorylcholines |
| | containing a C20:4 fatty acid unit. If detected with |
| | LC/MS, applying electro-spray ionization (ESI) mass |
| | spectrometry, the mass-to-charge ratio (m/z) of the |
| | positively charged ionic species is 544.4 Da (+/−0.5 |
| | Da). |
| Lysophosphatidylethanolamine | Lysophosphatidylethanolamine (C22:5) exhibits the |
| (C22:5) | following characteristic ionic species when detected |
| | with LC/MS, applying electro-spray ionization (ESI) |
| | mass spectrometry: mass-to-charge ratio (m/z) of the |
| | positively charged ionic species is 528.2 (+/−0.5). |
| Phosphatidylcholine | Phosphatidylcholine (C16:0/C16:0) represents the |
| (C16:0, C16:0) | sum parameter of glycerophosphorylcholines |
| | containing either the combination of of two C16:0 |
| | fatty acid units. The mass-to-charge ratio (m/z) of the |
| | ionised species is 734.8 Da (+/−0.5 Da). |
| Phosphatidylcholine | Phosphatidylcholine (C16:0, C20:5) exhibits the |
| (C16:0, C20:5) | following characteristic ionic species when detected |
| | with LC/MS, applying electro-spray ionization (ESI) |
| | mass spectrometry: mass-to-charge ratio (m/z) of the |
| | positively charged ionic species is 780.8 (+/−0.5). |
| Phosphatidylcholine | Phosphatidylcholine (C16:1, C18:2) represents the |
| (C16:1, C18:2) | sum parameter of glycerophosphorylcholines |
| | containing the combination of a C16:1 fatty acid unit |
| | and a C18:2 fatty acid unit. If detected with LC/MS, |
| | applying electro-spray ionization (ESI) mass |
| | spectrometry, the mass-to-charge ratio (m/z) of the |
| | positively charged ionic species is 756.8 Da (+/−0.5 |
| | Da). |
| Phosphatidylcholine | Phosphatidylcholine (C18:0, C18:1) represents the |
| (C18:0, C18:1) | sum parameter of glycerophosphorylcholines |
| | containing the combination of a C18:0 fatty acid unit |
| | and a C18:1 fatty acid unit. If detected with LC/MS, |
| | applying electro-spray ionization (ESI) mass |
| | spectrometry, the mass-to-charge ratio (m/z) of the |
| | positively charged ionic species is 788.6 Da (+/−0.5 |
| | Da). |
| Phosphatidylcholine | Phosphatidylcholine (C18:0, C18:2) represents the |
| (C18:0, C18:2) | sum parameter of glycerophosphorylcholines |
| | containing the combination of a C18:0 fatty acid unit |
| | and a C18:2 fatty acid unit. If detected with LC/MS, |
| | applying electro-spray ionization (ESI) mass |
| | spectrometry, the mass-to-charge ratio (m/z) of the |
| | positively charged ionic species is 786.6 Da (+/−0.5 |
| | Da). |
| Phosphatidylcholine | Phosphatidylcholine (C18:0, C20:3) exhibits the |
| (C18:0, C20:3) | following characteristic ionic species when detected |
| | with LC/MS, applying electro-spray ionization (ESI) |
| | mass spectrometry: mass-to-charge ratio (m/z) of the |
| | positively charged ionic species is 812.6 (+/−0.5). |
| Phosphatidylcholine | Phosphatidylcholine (C18:0, C20:4) represents the |
| (C18:0, C20:4) | sum parameter of glycerophosphorylcholines |
| | containing the combination of a C18:0 fatty acid unit |
| | and a C20:4 fatty acid unit. If detected with LC/MS, |
| | applying electro-spray ionization (ESI) mass |
| | spectrometry, the mass-to-charge ratio (m/z) of the |
| | positively charged ionic species is 810.8 Da (+/−0.5 |
| | Da). |
| Phosphatidylcholine | Phosphatidylcholine (C18:0, C22:6) represents the |
| (C18:0, C22:6) | sum parameter of glycerophosphorylcholines |
| | containing the combination of a C18:0 fatty acid unit |
| | and a C22:6 fatty acid unit. If detected with LC/MS, |
| | applying electro-spray ionization (ESI) mass |
| | spectrometry, the mass-to-charge ratio (m/z) of the |
| | positively charged ionic species is 834.8 Da (+/−0.5 |
| | Da). |
| Phosphatidylcholine | Phosphatidylcholine (C16:0/C20:3 C18:1/C18:2) |
| (C18:1, C18:2) | represents the sum parameter of |
| | glycerophosphorylcholines containing the |
| | combination of a C18:1 fatty acid unit and a C18:2 |
| | fatty acid unit. The mass-to-charge ratio (m/z) of the |
| | ionised species is 784.6 Da (+/−0.5 Da). |
| Phosphatidylcholine | Phosphatidylcholine (C16:0/C22:6 C18:2/C20:4) |
| (C18:2, C20:4) | represents the sum parameter of |
| | glycerophosphorylcholines containing either the |
| | combination of a C16:0 fatty acid unit and a C22:6 |
| | fatty acid unit or the combination of a C18:2 fatty acid |
| | unit and a C20:4 fatty acid unit. The mass-to-charge |
| | ratio (m/z) of the ionised species is 806.6 Da (+/−0.5 |
| | Da). |
| Phosphatidylcholine No 02 | Metabolite belongs to the class of |
| | glycerophosphocholines. It exhibits the following |
| | characteristic ionic species when detected with |
| | LC/MS, applying electro-spray ionization (ESI) mass |
| | spectrometry: mass-to-charge ratio (m/z) of the |
| | positively charged ionic species is 808.4 (+/−0.5). |
| Phosphatidylcholine No 04 | Metabolite belongs to the class of |
| | glycerophosphocholines. It exhibits the following |
| | characteristic ionic species when detected with |
| | LC/MS, applying electro-spray ionization (ESI) mass |
| | spectrometry: mass-to-charge ratio (m/z) of the |
| | positively charged ionic species is 796.8 (+/−0.5). |
| Sphingomyelin (d18:1, C23:0) | Sphingomyelin (d18:1, C23:0) exhibits the following |
| | characteristic ionic species when detected with |
| | LC/MS, applying electro-spray ionization (ESI) mass |
| | spectrometry: mass-to-charge ratio (m/z) of the |
| | positively charged ionic species is 801.8 (+/−0.5). |
| Sphingomyelin (d18:1, C24:0) | Sphingomyelin (d18:1, C24:0) represents the sum |
| | parameter of sphingomyelins containing the |
| | combination of a d18:1 long-chain base unit and a |
| | C24:0 fatty acid unit. If detected with LC/MS, |
| | applying electro-spray ionization (ESI) mass |
| | spectrometry, the mass-to-charge ratio (m/z) of the |
| | positively charged ionic species is 815.8 Da (+/−0.5 |
| | Da). |
| Sphingomyelin (d18:2, C16:0) | Sphingomyelin (d18:2, C16:0) exhibits the following |
| | characteristic ionic species when detected with |
| | LC/MS, applying electro-spray ionization (ESI) mass |
| | spectrometry: mass-to-charge ratio (m/z) of the |
| | positively charged ionic species is 723.6 (+/−0.5). |
| Sphingomyelin (d18:2, C18:0) | Sphingomyelin (d18:2, C18:0) exhibits the following |
| | characteristic ionic species when detected with |
| | LC/MS, applying electro-spray ionization (ESI) mass |
| | spectrometry: mass-to-charge ratio (m/z) of the |
| | positively charged ionic species is 729.8 (+/−0.5). |
| TAG (C16:0, C16:1) | Metabolite represents the sum of triacylglycerides |
| | containing the combination of a C16:0 fatty acid unit |
| | and a C16:1 fatty acid unit. It exhibits the following |
| | characteristic ionic species when detected with |
| | LC/MS, applying electro-spray ionization (ESI) mass |
| | spectrometry: mass-to-charge ratio (m/z) of the |
| | positively charged ionic species is 549.6 (+/−0.5). |
| TAG (C16:0, C18:1, C18:3) | TAG (C16:0, C18:1, C18:3) exhibits the following |
| | characteristic ionic species when detected with |
| | LC/MS, applying electro-spray ionization (ESI) mass |
| | spectrometry: mass-to-charge ratio (m/z) of the |
| | positively charged ionic species is 855.6 (+/−0.5). |
| TAG (C16:0, C18:2) | Metabolite represents the sum of triacylglycerides |
| | containing the combination of a C16:0 fatty acid unit |
| | and a C18:2 fatty acid unit. It exhibits the following |
| | characteristic ionic species when detected with |
| | LC/MS, applying electro-spray ionization (ESI) mass |
| | spectrometry: mass-to-charge ratio (m/z) of the |
| | positively charged ionic species is 575.6 (+/−0.5). |
| TAG (C18:1, C18:2) | Metabolite represents the sum of triacylglycerides |
| | containing the combination of a C18:1 fatty acid unit |
| | and a C18:2 fatty acid unit. It exhibits the following |
| | characteristic ionic species when detected with |
| | LC/MS, applying electro-spray ionization (ESI) mass |
| | spectrometry: mass-to-charge ratio (m/z) of the |
| | positively charged ionic species is 601.6 (+/−0.5). |
| TAG (C18:2, C18:2) | Metabolite represents the sum of triacylglycerides |
| | containing the combination of a C18:2 fatty acid unit |
| | and a C18:2 fatty acid unit. It exhibits the following |
| | characteristic ionic species when detected with |
| | LC/MS, applying electro-spray ionization (ESI) mass |
| | spectrometry: mass-to-charge ratio (m/z) of the |
| | positively charged ionic species is 599.6 (+/−0.5). |
| TAG (C18:2, C18:3) | Metabolite represents the sum of triacylglycerides |
| | containing the combination of a C18:2 fatty acid unit |
| | and a C18:3 fatty acid unit. It exhibits the following |
| | characteristic ionic species when detected with |
| | LC/MS, applying electro-spray ionization (ESI) mass |
| | spectrometry: mass-to-charge ratio (m/z) of the |
| | positively charged ionic species is 597.6 (+/−0.5). |
| TAG (DAG-Fragment) | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 600.6 (+/−0.5). |
| TAG No 01 | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 547.6 (+/−0.5). |
| TAG No 02 | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 695.6 (+/−0.5). |
| TAG No 05 | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 879.6 (+/−0.5). |
| TAG No 059 | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 904 (+/−0.5). |
| TAG No 07 | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 853.6 (+/−0.5). |

TABLE 11a
Markers for kidney toxicity (alpha 2 u globulin-nephropathy) in rats; Significant up-regulation changes in
bold (p-Value ≦ 0.1). All compounds were administers in high doses except for 1,1,2,2-tetrachloroethane.

| | 1,1,2,2- | | | 2,2,4- | | | | | | | | |
|---|---|---|---|---|---|---|---|---|---|---|---|---|
| | Tetrachloroethane | | | Trimethylpentane | | | D-Limonene | | | Decalin | | |
| Metabolite | m7 | m14 | m28 | m7 | m14 | m28 | m7 | m14 | m28 | m7 | m14 | m28 |
| Pantothenic acid | 4.46 | 4.32 | 4.32 | 2.59 | 3.07 | 3.03 | 1.04 | 1.85 | 1.76 | 2.13 | 3.31 | 3.02 |
| Oleic acid (C18:cis[9]1) | 1.11 | 1.18 | 1.18 | 2.10 | 2.37 | 2.97 | 1.78 | 1.84 | 2.35 | 3.11 | 4.04 | 6.72 |
| 4-Hydroxysphinganine | 2.03 | 2.04 | 2.04 | 1.46 | 1.34 | 1.26 | 1.40 | 1.29 | 1.41 | 2.00 | 1.98 | 1.84 |
| (t18:0, | | | | | | | | | | | | |
| Phytosphingosine), total | | | | | | | | | | | | |
| Nervonic acid | 1.09 | 0.96 | 0.96 | 1.60 | 1.38 | 1.22 | 1.40 | 1.28 | 1.47 | 2.01 | 2.23 | 1.74 |
| (C24:cis[15]1) | | | | | | | | | | | | |
| dihomo-gamma- | 2.56 | 3.40 | 3.40 | 2.42 | 2.06 | 2.77 | 1.66 | 1.62 | 1.82 | 2.56 | 2.86 | 2.61 |
| Linolenic acid | | | | | | | | | | | | |
| (C20:cis[8,11,14]3) | | | | | | | | | | | | |
| Ornithine | 2.10 | 2.38 | 2.38 | 1.12 | 1.23 | 1.23 | 1.22 | 1.46 | 1.60 | 1.21 | 1.11 | 1.16 |
| Glutamate | 2.31 | 1.80 | 1.80 | 1.57 | 1.44 | 1.74 | 1.33 | 1.31 | 1.45 | 1.47 | 1.66 | 1.97 |
| Xylitol | 12.21 | 10.32 | 10.32 | 1.65 | 1.33 | 1.68 | 3.32 | 2.56 | 3.04 | 2.95 | 2.18 | 1.48 |
| 3-Hydroxyindole | 1.39 | 2.05 | 2.05 | 1.92 | 2.88 | 2.30 | 1.80 | 1.32 | 1.68 | 1.96 | 1.80 | 2.69 |
| Glucuronic acid | 3.97 | 1.20 | 1.20 | 3.88 | 3.87 | 3.46 | 2.52 | 2.42 | 1.71 | 2.65 | 3.04 | 2.79 |
| Pseudouridine | 1.17 | 1.51 | 1.51 | 1.23 | 1.31 | 1.28 | 1.21 | 1.15 | 1.10 | 1.32 | 1.51 | 1.65 |
| Sphingomyelin | 1.65 | 1.34 | 1.34 | 1.62 | 1.59 | 1.54 | 1.22 | 1.30 | 1.26 | 1.47 | 1.82 | 1.43 |
| (d18:1, C16:0) | | | | | | | | | | | | |
| Sphingomyelin | 1.22 | 0.93 | 0.93 | 1.35 | 1.28 | 1.23 | 1.13 | 1.24 | 1.23 | 1.40 | 1.54 | 1.47 |
| (d18:2, C18:0) | | | | | | | | | | | | |
| Phosphatidylcholine | 1.48 | 1.21 | 1.21 | 1.35 | 1.34 | 1.35 | 1.63 | 1.58 | 1.87 | 1.75 | 2.03 | 1.84 |
| (C18:0, C18:1) | | | | | | | | | | | | |

TABLE 11b
Markers for kidney toxicity (alpha 2 u globulin-nephropathy) in rats; Significant down-regulation changes
in bold (p-Value ≦ 0.1). All compounds were administers in high doses except for 1,1,2,2-tetrachloroethane.

| | 1,1,2,2- | | | 2,2,4- | | | | | | | | |
|---|---|---|---|---|---|---|---|---|---|---|---|---|
| | Tetrachloroethane | | | Trimethylpentane | | | D-Limonene | | | Decalin | | |
| Metabolite | m7 | m14 | m28 | m7 | m14 | m28 | m7 | m14 | m28 | m7 | m14 | m28 |
| Choline plasmalogen No | 1.04 | 1.13 | 1.13 | 0.72 | 0.73 | 0.81 | 0.88 | 0.96 | 0.68 | 0.67 | 0.80 | 0.78 |
| 02 (putative) | | | | | | | | | | | | |
| Lysophosphatidylcholine | 0.63 | 0.89 | 0.89 | 0.72 | 0.73 | 0.80 | 0.80 | 0.93 | 0.87 | 0.84 | 0.83 | 0.87 |
| (C17:0) | | | | | | | | | | | | |
| Lyso PE (C22:0) | 0.43 | 0.65 | 0.65 | 0.54 | 0.49 | 0.45 | 0.75 | 0.78 | 0.69 | 0.82 | 0.76 | 0.71 |
| (putative) | | | | | | | | | | | | |
| TAG (C42:9) (DAG- | 0.76 | 0.72 | 0.72 | 0.67 | 0.64 | 0.76 | 1.12 | 0.76 | 0.92 | 0.71 | 0.38 | 0.38 |
| Fragment) (putative) | | | | | | | | | | | | |

1-20. (canceled)

21. A method for diagnosing kidney toxicity comprising:
(a) determining the amount of at least one biomarker selected from any one of Tables 1a, 1b, 1c, 1d, 2a, 2b, 2c, 2d, 3a, 3b, 3c, 3d, 4a, 4b, 4c, 4d, 5a, 5b, 6a, 6b, 7a, 7b, 8a, 8b, 11a or 11b in a test sample of a subject suspected to suffer from kidney toxicity, and
(b) comparing the amounts determined in step (a) to a reference, whereby kidney toxicity is to be diagnosed.

22. The method of claim 21, wherein said subject has been brought into contact with a compound suspected to be capable of inducing kidney toxicity.

23. The method of claim 22, wherein said compound is at least one compound selected from the group consisting of: Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-tetrachloroethane, 2,2,4-trimethylpentane, D-Limonene and Decalin.

24. The method of claim 21, wherein said reference is derived from (i) a subject or group of subjects which suffers from kidney toxicity or (ii) a subject or group of subjects which has been brought into contact with at least one compound selected from the group consisting of: Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-tetrachloroethane, 2,2,4-trimethylpentane, D-Limonene and Decalin.

25. The method of claim 24, wherein essentially identical amounts for the biomarkers in the test sample and the reference are indicative for kidney toxicity.

26. The method of claim 21, wherein said reference is derived from (i) a subject or group of subjects known to not suffer from kidney toxicity or (ii) a subject or group of subjects which has not been brought into contact with at least one compound selected from the group consisting of: Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-tetrachloroethane, 2,2,4-trimethylpentane, D-Limonene and Decalin.

27. The method of claim 21, wherein said reference is a calculated reference for the biomarkers for a population of subjects.

28. The method of claim 27, wherein amounts for the biomarkers which differ in the test sample in comparison to the reference are indicative for kidney toxicity.

29. A method of determining whether a compound is capable of inducing kidney toxicity in a subject comprising:
(a) determining in a sample of a subject which has been brought into contact with a compound suspected to be capable of inducing kidney toxicity the amount of at least one biomarker selected from any one of Tables 1a, 1b, 1c, 1d, 2a, 2b, 2c, 2d, 3a, 3b, 3c, 3d, 4a, 4b, 4c, 4d, 5a, 5b, 6a, 6b, 7a, 7b, 8a, 8b, 11a or 11b and
(b) comparing the amounts determined in step (a) to a reference, whereby the capability of the compound to induce kidney toxicity is determined.

30. The method of claim 29, wherein said compound is at least one compound selected from the group consisting of: Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-tetrachloroethane, 2,2,4-trimethylpentane, D-Limonene and Decalin.

31. The method of claim 29, wherein said reference is derived from (i) a subject or group of subjects which suffers from kidney toxicity or (ii) a subject or group of subjects which has been brought into contact with at least one compound selected from the group consisting of: Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-tetrachloroethane, 2,2,4-trimethylpentane, D-Limonene and Decalin.

32. The method of claim 31, wherein essentially identical amounts for the biomarkers in the test sample and the reference are indicative for kidney toxicity.

33. The method of claim 29, wherein said reference is derived from (i) a subject or group of subjects known to not suffer from kidney toxicity or (ii) a subject or group of subjects which has not been brought into contact with at least one compound selected from the group consisting of: Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-tetrachloroethane, 2,2,4-trimethylpentane, D-Limonene and Decalin.

34. The method of claim 29, wherein said reference is a calculated reference for the biomarkers for a population of subjects.

35. The method of claim 34, wherein amounts for the biomarkers which differ in the test sample in comparison to the reference are indicative for kidney toxicity.

36. A method of identifying a substance for treating kidney toxicity comprising the steps of:
(a) determining in a sample of a subject suffering from kidney toxicity which has been brought into contact with a candidate substance suspected to be capable of treating kidney toxicity the amount of at least one biomarker selected from any one of Tables 1a, 1b, 1c, 1d, 2a, 2b, 2c, 2d, 3a, 3b, 3c, 3d, 4a, 4b, 4c, 4d, 5a, 5b, 6a, 6b, 7a, 7b, 8a, 8b, 11a or 11b; and
(b) comparing the amounts determined in step (a) to a reference, whereby a substance capable of treating kidney toxicity is to be identified.

37. The method of claim 36, wherein said reference is derived from (i) a subject or group of subjects which suffers from kidney toxicity or (ii) a subject or group of subjects which has been brought into contact with at least one compound selected from the group consisting of: Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., and Tricresyl phosphate, 1,1,2,2-tetrachloroethane, 2,2,4-trimethylpentane, D-Limonene and Decalin.

38. The method of claim 37, wherein amounts for the biomarkers which differ in the test sample and the reference are indicative for a substance capable of treating kidney toxicity.

39. The method of claim 36, wherein said reference is derived from (i) a subject or group of subjects known to not suffer from kidney toxicity or (ii) a subject or group of subjects which has not been brought into contact with at least one compound selected from the group consisting of: Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-tetrachloroethane, 2,2,4-trimethylpentane, D-Limonene and Decalin.

40. The method of claim 36, wherein said reference is a calculated reference for the biomarkers in a population of subjects.

41. The method of claim 39, wherein essentially identical amounts for the biomarkers in the test sample and the reference are indicative for a substance capable of treating kidney toxicity.

42. A device for diagnosing kidney toxicity in a sample of a subject suspected to suffer therefrom comprising:
(a) an analyzing unit comprising a detection agent for at least one biomarker selected from any one of Tables 1a, 1b, 1c, 1d, 2a, 2b, 2c, 2d, 3a, 3b, 3c, 3d, 4a, 4b, 4c, 4d, 5a, 5b, 6a, 6b, 7a, 7b, 8a, 8b, 11a or 11b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby kidney toxicity is diagnosed.

43. The device of claim 42, wherein said stored reference is a reference derived from a subject or a group of subjects known to suffer from kidney toxicity or a subject or group of subjects which has been brought into contact with at least one compound selected from the group consisting of Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-tetrachloroethane, 2,2,4-trimethylpentane, D-Limonene and Decalin and said data processor executes instructions for comparing the amount of the at least one biomarker determined by the analyzing unit to the stored reference, wherein an essentially identical amount of the at least one biomarker in the test sample in comparison to the reference is indicative for the presence of kidney toxicity or wherein an amount of the at least one biomarker in the test sample which differs in comparison to the reference is indicative for the absence of kidney toxicity.

44. The device of claim 42, wherein said stored reference is a reference derived from a subject or a group of subjects known to not suffer from kidney toxicity or a subject or group of subjects which has not been brought into contact with at least one compound selected from the group consisting of Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-tetrachloroethane, 2,2,4-trimethylpentane, D-Limonene and Decalin and said data processor executes instructions for comparing the amount of the at least one biomarker determined by the analyzing unit to the stored reference, wherein an amount of the at least one biomarker in the test sample which differs in comparison to the reference is indicative for the presence of kidney toxicity or wherein an essentially identical amount of the at least one biomarker in the test sample in comparison to the reference is indicative for the absence of kidney toxicity.

45. A kit for diagnosing kidney toxicity comprising a detection agent for the at least one biomarker selected from any one of Tables 1a, 1b, 1c, 1d, 2a, 2b, 2c, 2d, 3a, 3b, 3c, 3d, 4a, 4b, 4c, 4d, 5a, 5b, 6a, 6b, 7a, 7b, 8a, 8b, 11a or 11b and standards for the at least one biomarker the concentration of which is derived from (i) a subject or a group of subjects known to suffer from kidney toxicity or a subject or group of subjects which has been brought into contact with at least one compound selected from the group consisting of Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-tetrachloroethane, 2,2,4-trimethylpentane, D-Limonene and Decalin or derived (ii) from a subject or a group of subjects known to not suffer from kidney toxicity or a subject or group of subjects which has not been brought into contact with at least one compound selected from the group consisting of Amphotericin B, Beta-ionone, Caffeine, Captopril, Carboplatin, Cyclosporin A, Dichlorprop-p, Dipyrone, Ethylbenzene, Furosemide, Hexachlorobutadiene, Hydroquinone, Lisinopril, Lithocholic acid, MCPA, Mecoprop-p, Penicillamine, Pentachlorophenol, Probenecid, Ramipril, Theobromine, Theophylline, Tobramycin s.c., Tricresyl phosphate, 1,1,2,2-tetrachloroethane, 2,2,4-trimethylpentane, D-Limonene and Decalin.
